(19) United States
(12) Patent Application Publication
(10) Pub. No.: US 2007/0042981 A1
(43) Pub. Date: Feb. 22, 2007

(54) Use of eukaryotic genes affecting chromatin separation for diagnosis and treatment of proliferative diseases

(75) Inventors: Christophe Echeverri, Dresden (DE); Anthony Hyman, Dresden (DE); Pierre Gonczy, Lausanne (CH); Birte Sonnichsen, Dresden (DE); Steven Jones, Vancouver (CA); Andrew Walsh, Dresden (DE); Liisa Koski, Dresden (DE)

Correspondence Address: CONNOLLY BOVE LODGE & HUTZ, LLP, P O BOX 2207, WILMINGTON, DE, 19899, US

(73) Assignee: Cenix BioScience GmbH, Dresden (DE)

(21) Appl. No.: 10/572,123

(22) PCT Filed: Sep. 15, 2004
(86) PCT No.: PCT/EP04/10311

§ 371 Date: Sep. 15, 2006

Related U.S. Application Data
(60) Provisional application No. 60/502,653, filed on Sep. 15, 2003.

(51) Int. Cl. A61K 48/00; A61K 39/395; A61K 38/55; C12Q 1/68; G01N 33/574

(52) U.S. Cl. 514/44; 514/12; 424/155.1; 435/6; 435/7.23

(57) ABSTRACT

The present invention relates to the significant functional role of several C. elegans genes and of their corresponding gene products in chromatin separation during cell division that could be identified by means of RNA-mediated interference (RNAi) and to the identification and isolation of functional orthologs of said genes including all biologically functional derivatives thereof. The invention further relates to the use of said genes and gene products (including said orthologs) in the development or isolation of anti-proliferative agents, particularly their use in appropriate screening assays, and their use for diagnosis and treatment of proliferative and other diseases. In particular, the invention relates to the use of small interfering RNAs derived from said genes for the treatment of proliferative diseases.

The present invention relates to the use of agents interfering with chromatin separation for the treatment of diseases, especially proliferative diseases.

Metazoan cell division (mitosis) consists of an extremely complex, highly regulated set of cellular processes which must be tightly co-ordinated, perfectly timed, and closely monitored in order to ensure the correct delivery of cellular materials to daughter cells. Defects in these processes are known to cause a wide range of so-called proliferative diseases, including all forms of cancer. Since cell division represents one of the few, if not the only cellular process that is common to the aetiology of all forms of cancer, its specific inhibition has long been recognised as a preferred site of therapeutic intervention.

Although mitotic inhibitor drugs are recognised as one of the most promising classes of chemotherapeutic agents, screening attempts to find new drug candidates in this class have been undermined by the strong inherent tendency of such screens to identify agents that target a single protein, tubulin. Tubulin polymerises to form microtubules, the primary cytoskeletal elements needed for mitotic spindle function and chromosome segregation. Microtubules as such, however, are ubiquitously needed in almost all cell types, whether dividing or not, a fact which therefore explains many of the unwanted side effects caused by anti-tubulin drugs.

Perhaps the best known example of a highly successful anti-neoplastic drug that targets tubulin is paclitaxel, and its marketed derivative, Taxol. Its applicability has indeed been seriously limited by difficulties in determining an adequate dosing regimen due to a range of problematic side effects. Taxol treatment has resulted in anaphylaxis and severe hypersensitivity reactions characterised by dyspnea and hypotension requiring treatment, angioedema, and generalised urticaria in 2-4% of patients in clinical trials. Although Taxol is administered after pretreatment with corticosteroids, fatal reactions have occurred. Severe conductance abnormalities resulting in life-threatening cardiac arrhythmia occur in less than 1 percent of patients and must be treated by insertion of a pacemaker. Taxol can cause fetal harm or fetal death in pregnant women. Furthermore, administration is commonly accompanied by tachycardia, hypotension, flushing, skin reactions and shortness-of-breath (mild dyspnea). Reasons for these strong side-effects may be that since tubulin does not only play an essential role in spindle formation, but also plays significant roles in other cellular processes like for instance cytoskeleton generation and intracellular protein transport.

Consequently, although Taxol has been hailed by many as the most successful new anti-cancer therapeutic of the last three decades, there is still a need for anti-cancer drugs that do not show the disadvantages of Taxol.

Therefore, the problem underlying the present invention resides in providing improved potent anti-cancer drugs, particularly with less severe side effects.

The problem is solved by the use of an isolated nucleic acid molecule comprising a sequence selected from the group of sequences consisting of:
a) the nucleic acid sequences presented in SEQ ID NO. 21, 23, 1, 3, 5, 7, 9, 11, 13, 15, 17, 19, 25, 27, 29, 31, 32, 33, 34, 35, 36, 37, 38;
b) nucleic acid sequences encoding polypeptides that exhibit a sequence identity with the protein encoded by a nucleic acid according to a) of at least 25 % over 100 residues and/or which are detectable in a computer aided search using the BLAST sequence analysis programs with an e-value of at most 10⁻⁵,
c) sequences of nucleic acid molecules which are capable of hybridizing with the nucleic acid molecules with sequences corresponding to (a) or (b) under conditions of medium or high stringency,
d) the antisense-sequence of any of the sequences as defined in (a), (b) or (c),
e) fragments of (a), (b), (c) or (d),
f) double-stranded RNA or single-stranded RNA in the antisense or sense direction corresponding to any of the sequences as defined in (a), (b), (c), (d), or (e)
   for the manufacture of a medicament for the inhibition of chromatin separation.

The present invention is based on the concept to provide agents interfering with chromatin separation during cell division. Chromatin separation takes place during anaphase of mitosis and is an essential part of cell division.

Since chromatin separation—in contrast to microtubule formation—is a cell division-specific process, the inhibition of target proteins involved in chromatin separation results in an efficient impairment of mitosis as well as in a reduced number of side effects caused by the inhibition of other significant cellular processes.

The present invention discloses for the first time for a variety of proteins and genes that they are involved in chromatin separation during the anaphase of mitosis. This newly identified function of these target genes and their corresponding gene products, any homologs, orthologs and derivatives thereof enables their use in the development of a wide range of medicaments against proliferative diseases including cancer. These medicaments could be used in treatment of proliferative diseases, particularly in those cases where the disorder relates to cell division, regulation of cell division, or is dependent on chromatin separation during cell division. Furthermore, the newly identified function enables the use in diagnosis and the development of diagnostic agents.

For the identification of target genes being involved in chromatin separation, a large-scale RNAi technique-based screen was performed for 19514 (that means 99.7%) of the predicted open reading frames in the C. elegans genome. For the performance of this large-scale screen double-stranded RNA corresponding to the individual open reading frames was produced and micro-injected into adult C. elegans hermaphrodites, and the resulting embryos were analysed 24 hours later using time-lapse DIC microscopy.

The nematode C. elegans exhibits an almost entirely translucent body throughout its development, thereby offering unparalleled microscopic access for exquisitely detailed cytological documentation, even for the earliest steps of embryogenesis. This important feature, along with its short life cycle (3-5 days), its ease of cultivation, and its low maintenance costs, has helped make C. elegans arguably the best studied of all metazoans. Also, sequence data are now available for over 97% of the C. elegans genome (C. elegans Sequencing Consortium. Genome sequence of the nematode C. elegans: a platform for investigating biology. Science 282, 2012-2018 (1998)). Thus, C. elegans is an ideal organism for applying the new technique of RNA-mediated interference (RNAi). This technique consists in the targeted, sequence-specific inhibition of gene expression, as mediated by the introduction into an adult worm of double-stranded RNA (dsRNA) molecules corresponding to portions of the coding sequences of interest (Fire et al., Potent and specific genetic interference by double-stranded RNA in Caenorhabditis elegans. Nature 391, 806-811 (1998)). For the vast majority of C. elegans genes tested to date, this has been shown to yield a sequence-specific inhibition of the targeted gene's expression, accompanied by clearly detectable loss of function phenotypes in the treated worm's F1 progeny (and even in some cases, in the treated worm itself).

In the context of the present invention, a screening assay in C. elegans based on ‘genomic RNA mediated interference (RNAi)’ combined with a highly probative microscopic assay for documenting the first rounds of embryonic cell division was used (Sulston et al., The embryonic cell lineage of the nematode Caenorhabditis elegans. Dev. Biol. 100, 64-119 (1983); Gönczy et al., Dissection of cell division processes in the one cell stage Caenorhabditis elegans embryo by mutational analysis. J Cell Biol 144, 927-946 (1999)). With this combination of techniques a selected gene and also a variety of selected genes can be functionally characterized with unprecedented speed and efficiency.

The DIC microscopy generated movies were analyzed to identify those samples whereby cell division was altered or disrupted. In order to perform the analysis in a robust, consistent and reproducible fashion, each movie was analyzed with regard to 47 different parameters. In other words, 47 features of normal cell division (i.e. cell division in wild type worms) were scored for every RNAi phenotype generated by the genome-wide application of RNAi across the entire C. elegans genome.

A powerful confirmation and validation of the DIC assay, and the depth of information that the assays yield, was that equivalent phenotypes were found to represent closely related proteins, proteins within the same family or finctionally equivalent proteins. In other words, if the RNAi-induced phenotypes of two separately analyzed genes are the same, it is very likely that the two proteins are either within the same protein class or share a similar function or at the very least, are both involved in the same biological mechanism or process. Therefore, the screen can be used to class or group proteins according to their function. Consequently, any genes that give rise to similar RNAi phenotypes are related and are justified to be considered within single functional classes.

“Nucleic acids” according to the present invention comprises all known nucleic acids such as DNA, RNA, peptide nucleic acids, morpholinos, and nucleic acids with backbone structures other than phosphodiesters, such as phosphothiates or phosphoramidates.

“Ihibition of chromatin separation” according to the present invention includes halting or arresting as well as retarding or slowing down of chromatin separation.

In a preferred embodiment of the invention, the nucleic acid molecule comprises a nucleic acid molecule with a sequence selected from the group of sequences as presented in SEQ ID NO. 21, 23, 1, 3, 5, 7, 9, 11, 13, 15, 17, 19, 25, 27, 29, 31, 33, 35, 37. Preferably, the nucleic acid molecule consists of a nucleic acid molecule with a sequence selected from said group of sequences.

The term “comprise” preferably refers to nucleic acids, or nucleic acids comprising a sequence, in which the nucleic acids with the described sequences are functionally relevant, e.g. for diagnostic use or therapeutic use, such as vectors for therapeutical use or expression of corresponding RNAs or proteins. Preferably, any additional nucleic acids upstream or downstream of the sequence are not longer than 20 kb. More preferred, the term “comprise” does not relate to large constructs accidentally including the sequence, uch as genomic BAC or YAC clones.

In detail, the individual SEQ ID No. denotes the following sequences:
SEQ ID NO. 1 the nucleotide sequence of the C. elegans gene F55C5.4 (Worrnbase accession No. CE11156)
SEQ ID NO. 2 the deduced amino acid sequence of the C. elegans gene F55C5.4 (Wormbase accession No. CE 11156)
SEQ ID NO. 3 the nucleotide sequence of the human ortholog of F55C5.4 (GenBank accession No. NP_{—}017760)
SEQ ID NO. 4 the deduced amino acid sequence of the human ortholog of F55C5.4 (GenBank accession No. NP_{—}060230)
SEQ ID NO. 5 the nucleotide sequence of the C. elegans gene C33D12.3 (Wormbase accession No. CE06905)
SEQ ID NO. 6 the deduced amino acid sequence of the C. elegans gene C33D12.3 (Wormbase accession No. CE06905)
SEQ ID NO. 7 the nucleotide sequence of the human homolog of C33D12.3 (GenBank accession No. NP_{—}055032)
SEQ ID NO. 8 the deduced amino acid sequence of the human homolog of C33D12.3 (GenBank accession No. NP_{—}055032)
SEQ ID NO. 9 the nucleotide sequence of the mouse homolog of C33D12.3 (GenBank accession No. XP_{—}109361)
SEQ ID NO. 10 the deduced amino acid sequence of the mouse homolog of C33D12.3 (GenBank accession No. XP_{—}109361)
SEQ ID NO. 11 the nucleotide sequence (corresponding to the mRNA) of the rat homolog of C33D 12.3 (GenBank accession No. NM_{—}172042)
SEQ ID NO. 12 the deduced amino acid sequence of the rat homolog of C33D12.3 (GenBank accession No. NP_{—}742039)
SEQ ID NO. 13 the nucleotide sequence of the Drosophila homolog of C33D12.3 (GenBank accession No. AAF47456)
SEQ ID NO. 14 the deduced amino acid sequence of the Drosophila homolog of C33D12.3 (GenBank accession No. AAF47456)
SEQ ID NO. 15 the nucleotide sequence of the C. elegans gene K12D12.2 (Wormbase accession No. CE02271)
SEQ ID NO. 16 the deduced amino acid sequence of the C. elegans gene K12D12.2 (Wormbase accession No. CE02271)
SEQ ID NO. 17 the nucleotide sequence of the human ortholog of K12D12.2 (GenBank accession No. XM_{—}058073)
SEQ ID NO. 18 the deduced amino acid sequence of the human ortholog of K12D12.2 (GenBank accession No. XP_{—}058073)
SEQ ID NO.19 the nucleotide sequence of the Drosophila ortholog of K12D12.2 (GenBank accession No. AE003513)
SEQ ID NO. 20 the deduced amino acid sequence of the Drosophila ortholog of K12D12.2 (GenBanlk accession No. AAF49028)
SEQ ID NO. 21 the nucleotide sequence of the C. elegans gene Y48E1B.12 (Wormbase accession No. CE14874)
SEQ ID NO. 22 the deduced amino acid sequence of the C. elegans gene Y48E1B.12 (Wormbase accession No. CE14874)
SEQ ID NO. 23 the nucleotide sequence of the human homolog of Y48E1B.12 (GenBank accession No. XP_{—}027054)
SEQ ID NO. 24 the deduced amino acid sequence of the human homolog of Y48E1B.12 (GenBank accession No. XP_{—}027054)
SEQ ID NO. 25 the nucleotide sequence of the C. elegans gene Y110A7A.1a (Wormbase accession No. CE24117) SEQ ID NO. 26 the deduced amino acid sequence of the C. elegans gene Y110A7A.1a (Wormbase accession No. CE24117)
SEQ ID NO. 27 the nucleotide sequence of the human homolog of Y110A7A.1a (GenBank accession No. XP_{—}166201)
SEQ ID NO. 28 the deduced amino acid sequence of the human homolog Y110A7A.1a (GenBank accession No. XP_{—}166201)
SEQ ID NO. 29 the nucleotide sequence of the C. elegans gene W02D9.2 (Wormbase accession No. CE14456)
SEQ ID NO. 30 the deduced amino acid sequence of the C. elegans gene W02D9.2 (Wormbase accession No. CE14456)
SEQ ID NO. 31 the nucleotide sequence of the human ortholog of W02D9.2 (GenBank accession No. BC012469)
SEQ ID NO. 32 the deduced amino acid sequence of the human ortholog of W02D9.2 (GenBank accession No. AAH12469)
SEQ ID NO. 33 the nucleotide sequence of the human homolog of W02D9.2 (GenBank accession No. NP_{—}115688)
SEQ ID NO. 34 the deduced amino acid sequence of the human homolog of W02D9.2 (GenBank accession No. NP_{—}115688)
SEQ ID NO. 35 the nucleotide sequence of the Drosophila ortholog of W02D9.2 (GenBank accession No. AAF51019)
SEQ ID NO. 36 the deduced amino acid sequence of the Drosophila ortholog of W02D9.2 (GenBank accession No. AAF51019)
SEQ ID NO. 37 the nucleotide sequence of the yeast homolog of W02D9.2 (GenBank accession No. NC_{—}001139.3, base pairs 842847 to 843593)
SEQ ID NO. 38 the deduced amino acid sequence of the yeast homolog of W02D9.2 (GenBank accession No. NP_{—}011688)

Unless otherwise specified, the manipulations of nucleic acids and polypeptides/-proteins can be performed using standard methods of molecular biology and immunology (see, e.g. Maniatis et al. (1989), Molecular cloning: A laboratory manual, Cold Spring Harbor Lab., Cold Spring Harbor, N.Y.; Ausubel, F. M. et al. (eds.) “Current protocols in Molecular Biology”. Joln Wiley and Sons, 1995; Tijssen, P., Practice and Theory of Enzyme Inmunoassays, Elsevier Press, Amsterdam, Oxford, New York, 1985).

The present invention describes genes identified as having essential functions in cell division in the model organism C. elegans. The basis for performing research in model organisms is that the newly discovered functions for the genes in C. elegans will be conserved in other species including humans. Cell division and chromatin separation during cell division is highly conserved during evolution and therefore the approach of discovering a gene function in C. elegans and using the information to characterise or assign functions for human homologs or orthologs is well justified.

One theme of conservation is that the gene function can be conserved with substantial divergence of sequence. In the present invention this theme of conservation is not defined. However, if other genes are discovered to have finctions that result in the gene product being identified as the same gene product as those claimed in the present invention then the present claims also apply to such genes.

However, the most frequent theme of conservation of genes during evolution is that the gene sequence may be conserved. This theme of conservation is particularly frequent for genes involved in highly conserved processes such as cell division. This means that the DNA nucleotide sequence or the protein coding sequence of the gene are very similar in different species, which in turn suggests that the function of the gene is the same in the different species.

Therefore, in a further preferred embodiment, the nucleic acid molecule has a sequence that encodes a polypeptide exhibiting a sequence identity with a protein encoded by SEQ ID NO. 21, 23, 1, 3, 5, 7, 9, 11, 13, 15, 17, 19, 25, 27, 29, 31, 33, 35, 37 of at least 25 % over 100 residues, preferably of at least 30 % over 100 residues, more preferably of at least 50 % over 100 residues, particularly of at least 70 % over 100 residues on amino acid level.

These very high sequence similarities are usually shown by polypeptides which are orthologs or homologs of the above sequences. A homolog is a protein with similar sequence from the same or another species (an homolog's sequence similarity originates from a speciation event or from a gene duplication, i.e. a homolog is a related protein in any species or the same protein in another species). A subgroup of homologs are defined as orthologs. An ortholog is essentially the same protein as the one it is compared to, but it is derived from another species (an ortholog's sequence similarity originates from a speciation event rather than a gene duplication). It is known to a person skilled in the art, that in a conserved process such as cell division, homologous and orthologous proteins, particularly orthologous proteins, are very likely to serve the same biological function. In the present case, the most relevant biological function is the involvement in, particularly the requirement for, chromatin separation during cell division.

Advantageously, it could be shown that human orthologs of the C.elegans genes identified in the context of this invention are required for proliferation, cell survival and mitosis (see Example 10). This finding indicates that the human orthologs are required for chromatin separation during cell division and can be used in the context of diagnosis and treatment of proliferative diseases.

The person skilled in the art is familiar with different methods and criteria to identify homologs and orthologs. In the context of the present invention, homologs and orthologs were identified based on sequence similarity according to the procedure described in Example 9.

The nucleic acid molecule may also comprise a sequence that is detectable in a computer aided database search/alignment with an e-value of at most 10⁻⁵, preferably with an e-value of at most 10⁻¹², particularly with an e-value of at most 10⁻²⁰ or fragments thereof, whereby the database sequences are compared to the sequences as defined under a). The nucleic acid molecule may also comprise a sequence that is considered an ortholog according to the criteria of the present invention (see Example 9). Generally, the grade of sequence identity can be calculated by any software program that is capable to perform protein sequence alignments known in the art. Hereby it is also included that identical amino acid regions are interrupted by gaps that can be variable in their length.

For this kind of analysis or alignments the “BLAST sequence analysis programs” are particularly preferred. The “BLAST sequence analysis programs” which may be used for sequence analysis are publically available and known to anyone skilled in the art. Known analysis programs for sequence alignments, particularly the “BLAST sequence analysis programs”, calculate so called “e-values” to characterize the grade of homology between the compared sequences. Generally, a small e-value characterizes a high sequence similarity, whereas larger e-values characterize lower sequence similarity.

The degree of similarity required for the sequence variant will depend upon the intended use of the sequence. It is well within the capability of a person skilled in the art to effect mutational, insertional and deletional mutations which are designed to improve the function of the sequence or otherwise provide a methodological advantage.

The aforementioned grades of sequence identities with proteins encoded by the above SEQ IDs are characteristic for such polypeptides that are strongly homologous to the above sequences, in particular for polypeptides that are “orthologous” or “homologous” to the polypeptides of a).

Table 1 shows the e-values that have been calculated for the alignments on amino acid level with homologs and orthologsof the corresponding C. elegans gene. Hereby, e-values lower than 10⁻⁵ on amino acid level characterize homologs of the corresponding C. elegans genes. If the C. elegans gene is itself a reciprocal hit of the identified homolog with an e-value of less than 10⁻⁵, then the homolog is identified as an ortholog (see also Example 9).

TABLE 1
Sequence similarities between the C. elegans genes
F55C5.4, C33D12.3, K12D12.2, Y48E1B.12, Y110A7A.1a,
W02D9.2 and their human, mouse, rat, Drosophila,
and yeast homologs and orthologs.

| | e-value for the alignment with the C. elegans |
|---|---|
| C. elegans gene | gene on amino acid level |
| F55C5.4 | Human ortholog 4*10⁻¹⁹ |
| C33D12.3 | Human homolog 3*10⁻¹⁴ |
| | Mouse ortholog 4*10⁻¹⁹ |
| | Rat homolog 5*10⁻¹² |
| | Drosophila ortholog 9*10⁻¹⁷ |
| K12D12.2 | Human ortholog 5*10⁻²¹ |
| | Drosoph. ortholog 1*10⁻²⁹ |
| Y48E1B.12 | Human homolog 1*10⁻³ |
| Y110A7A.1a | Human ortholog 7*10⁻⁴⁰ |
| W02D9.2 | Human ortholog 6*10⁻⁴⁶ (GenBank Acc. No. |
| | AAH12469) |
| | Human homolog 2*10⁻¹⁴ (Gene Bank Acc. No. |
| | NP_115688) |
| | Drosophila ortholog 1*10⁻⁵⁰ |
| | Yeast ortholog 3*10⁻¹⁴ |

According to a further preferred embodiment, the nucleic acid molecule comprises a nucleotide sequence which is capable of hybridizing with the nucleic acid sequences of (a) or (b) under conditions of medium/high stringency.

In such hybrids, duplex formation and stability depend on substantial complementarity between the two strands of the hybrid and a certain degree of mismatch can be tolerated. Therefore, the nucleic acid molecules and probes of the present invention may include mutations (both single and multiple), deletions, insertions of the above identified sequences, and combinations thereof, as long as said sequence variants still have substantial sequence similarity to the original sequence which permits the formation of stable hybrids with the target nucleotide sequence of interest.

Suitable experimental conditions for determining whether a given DNA or RNA sequence “hybridizes” to a specified polynucleotide or oligonucleotide probe involve presoaking of the filter containing the DNA or RNA to examine for hybridization in 5×SSC (sodium chloride/sodium citrate) buffer for 10 minutes, and prehybridization of the filter in a solution of 5×SSC, 5×Denhardt's solution, 0.5% SDS and 100 mg/ml of denaturated sonicated salmon sperm DNA (Maniatis et al.,1989), followed by hybridization in the same solution containing a concentration of 10 ng/ml of a random primed (Feinberg, A. P. and Vogelstein, B. (1983), Anal. Biochem. 132:6-13), ³²P-dCTP-labeled (specific activity >1×10⁹ cpm/μg) probe for 12 hours at approximately 45° C. The filter is then washed twice for 30 minutes in 2×SSC, 0.5% SDS at at least 55° C. (low stringency), at least 60° C. (medium stringency), preferably at least 65° C. (medium/high stringency), more preferably at least 70° C. (high stringency) or most preferably at least 75° C. (very high stringency). Molecules to which the probe hybridizes under the chosen conditions are detected using an x-ray film or a “phosphor imager”.

According to a further preferred embodiment, the nucleic acid molecules may also have the antisense-sequence of any of the sequences as defined in (a), (b) or (c).

According to a further preferred embodiment, fragments of the nucleic acid molecules as described above may be used.

The term “fragment” as used according to the present invention can have different meanings depending on the molecule and purpose referred to. A person skilled in the art knows how to choose appropriate fragments for the relevant purpose. Preferably, a fragment should be specific for the sequence it is derived from. The meaning of the term “specific” is known in the art. Preferably, specific in this context means that in a BLAST search performed with the sequence fragment, the original sequence (from which the fragment is derived) would be identified with a lower e-value than all other sequences relevant in the context of the current use (e.g. all other sequences of nucleic acids present in the investigated sample). More preferably, the original sequence should be identified with the lowest e-value compared to all other sequences identified. Alternatively, “specific” means that, under the applied conditions, the fragment binds only to the nucleic acid molecule it is derived from. The criterion of specificity is usually achieved by fragments larger than 15 nucleotides, preferably larger than 19 nucleotides. Preferably, the fragments are chosen from sequence regions of high complexity. Low complexity regions can be identified by database searches or low complexity filters available in standard sequence analysis programs.

“Biologically active” fragments or derivatives can be generated by a person skilled in the art. Hereby, the fragments or derivatives should have a similar “biological function” as the nucleic acid they are derived from. According to the present invention the most relevant biological function is the involvement in, inhibition of, activation of, or requirement for chromatin separation during cell division.

The isolated nucleic acid molecules defined as under (a) to (e) may be used for influencing cell division and/or cell proliferation, particularly by inhibiting chromatin separation during cell division, either in vitro or in vivo.

Inhibition of chromatin separation using said nucleic acid molecules can be achieved by different ways familiar to the person skilled in the art. For example, the isolated nucleic acid molecules may be inserted downstream of a strong promotor to overexpress the corresponding protein or polypeptide. Overexpression of the protein or polypeptide may lead to suppression of the endogenous protein's biological function. By introducing deletions or.other mutations into the nucleic acids, or by using suitable fragments, it is s possible to generate sequences encoding dominant-negative peptides or polypeptides. Such dominant-negative peptides or polypeptides can inhibit the function of the corresponding endogenous protein.

Certain nucleic acids can be used to inhibit expression (transcription and/or translation) of the endogenous genes to inhibit chromatin separation. E.g. peptide nucleic acids comprising sequences as identified above can suppress expression of the corresponding endogenous gene by forming DNA triplex structures with the gene. Other nucleic acids, such as antisense morpholino oligonucleotides or ribozymes, can be used to interfere with RNA transcribed from the endogenous gene.

The application of automated gene synthesis provides an opportunity for generating sequence variants of the naturally occurring genes. It will be appreciated that polynucleotides coding for synthetic variants of the corresponding amino acid sequences can be generated which, for example, will result in one or more amino acids substitutions, deletions or additions. Also, nucleic acid molecules comprising one or more synthetic nucleotide derivatives (including morpholinos) which provide said nucleotide sequence with a desired feature, e.g. a reactive or detectable group, can be prepared. Synthetic derivatives with desirable properties may also be included in the corresponding polypeptides. All such derivatives and fragments of the above identified genes and gene products showing at least part of the biological activity or biological function of the naturally occurring sequences or which are still suitable to be used, for example, as probes for, e.g. identification of homologous genes or gene products, are included within the scope of the present invention. Also included are such derivatives and fragments whose activity or function is counteracting to the biological activity or biological function of the naturally occurring sequences, e.g. derivatives and fragments that encode dominant-negative molecules.

Having herein provided the nucleotide sequences of various genes fiuctionally involved in chromatin separation during cell division, it will be appreciated that automated techniques of gene synthesis and/or amplification may be used to isolate said nucleic acid molecules in vitro. Because of the length of some coding sequences, application of automated synthesis may require staged gene construction, in which regions of the gene up to about 300 nucleotides in length are synthesized individually and then ligated in correct succession for final assembly. Individually sythesized gene regions can be amplified prior to assembly, using polymerase chain reaction (PCR) technology. The technique of PCR amplification may also be used to directly generate all or part of the final genes/nucleic acid molecules. In this case, primers are synthesized which will be able to prime the PCR amplification of the final product, either in one piece or in several pieces that may be ligated together. For this purpose, either cDNA or genomic DNA may be used as the template for the PCR amplification. The cDNA template Mnay be derived from commercially available or self-constructed cDNA libraries.

According to a furter preferred embodiment, the invention relates to the use of the above identified nucleic acid molecules or fragments thereof in form of RNA, particularly antisense RNA and double-stranded RNA, for the manufacture of a medicament for the inhibition of chromatin separation. Also ribozymes can be generated for the above identified sequences and used to degrade RNA transcribed from the corresponding endogenous genes.

As stated above, double-stranded RNA oligonucleotides effect silencing of the expression of gene(s) which are highly homologous to either of the RNA strands in the duplex. Recent discoveries had revealed that this effect, called RNA interference (RNAi), that had been originally discovered in C. elegans, can also be observed in mammalian, particularly in human cells. Thus, inhibition of a specific gene function by RNA interference can also be performed in mammalian cells, particularly also in human cells.

As shown in FIG. 1, the inhibition of a nucleic acid molecule as defined under (a) to (f) by RNAi in C. elegans inhibits cell division by impairing chromatin separation during anaphase of mitosis.

Particularly preferred is the use of these RNA molecules in a therapeutical application of the RNAi technique, particularly in humans or in human cells.

An RNAi technique particularly suited for mammalian cells makes use of double-stranded RNA oligonucleotides known as “small interfering RNA” (siRNA).

Therefore, according to a further preferred embodiment, the invention relates to the use of nucleic molecules comprising small interfering RNA with a sequence corresponding to any of the sequences identified above.

These siRNA molecules can be used for the therapeutical silencing of the expression of the genes of the invention comprising nucleic acid sequences as defined under (a) to (f), in mammalian cells, particularly in human cells, particularly for the therapy of a proliferative disease.

The inhibition of a specific target gene in mammals is achieved by the introduction of an siRNA-molecule having a sequence that is specific (see above) for the target gene into the mammalian cell. The siRNAs comprise a first and a second RNA strand, both hybridized to each other, wherein the sequence of the first RNA strand is a fragment of one of the sequences as defined in a) to f) and wherein the sequence of the second RNA strand is the antisense-strand of the first RNA strand. The siRNA-molecules may possess a characteristic 2- or 3-nucleotide 3'-overhanging sequence. Each strand of the siRNA molecule preferably has a length of 19 to 31 nucleotides.

The siRNAs can be introduced into the mammalian cell by any suitable known method of cell transfection, particularly lipofection, electroporation or microinjection. The RNA oligonucleotides can be generated and hybridized to each other in vitro or in vivo according to any of the known RNA synthesis methods.

The possibility to inhibit gene expression of disease-associated genes also in mammalian cells and in particular in human cells, make siRNAs or vector systems capable of producing siRNAs, having the sequence of those disease-associated genes, an interesting therapeutical agent for pharmaceutical compositions. Particularly siRNAs having sequences as defined in the present invention or that are homologous or orthologous to one of those genes can be used for the manufacture of medicaments for the inhibition of chromatin separaration and for the therapy of diseases, particularly proliferative diseases (see above). Similarly, nucleic acid vectors capable of producing those siRNAs can be used for the manufacture of such medicaments.

In another embodiment, the invention relates to the use of a nucleic acid molecule as defined above, wherein the nucleic acid molecule is contained in at least one nucleic acid expression vector which is capable of producing a double-stranded RNA-molecule comprising a sense-RNA-stand and an antisense-RNA-strand under suitable conditions, wherein each RNA-strand, independently from the other, has a length of 19 to 31 nucleotides.

In this alternative method (also described in Tuschl, Nature Biotechnology, Vol. 20, pp. 446-448), vector systems capable of producing siRNAs instead of the siRNAs themselves are introduced into the mammalian cell for downregulating gene expression.

The preferred lengths of the RNA-strands produced by such vectors correspond to those preferred for siRNAs in general (see below).

“Suitable conditions” for the production of the above double-stranded RNA-molecule are all in vivo or in vitro conditions that according to the state of art allow the expression of a first and a second RNA-strand with the above sequences and lengths that—when hybridized—form a double-stranded RNA-molecule. Particularly preferred “suitable conditions” for the production of the above double-stranded RNA-molecule are the “in vivo conditions” in a living human or animal cell or the “in vitro conditions” in cultured human or animal cells.

The “nucleic acid expression vector” may be an extrachromosomal entity, the replication of which is independent of chromosomal replication, e.g. a plasmid. Alternatively, the vector may be one which, when introduced into a host cell, particularly into a mammalian host cell, is integrated into the host cell genome and replicated together with the chromosome(s) into which it has been integrated. Preferably, the “nucleic acid expression vector” may be an expression vector which is usually applied in gene therapeutic methods in humans, particularly a retroviral vector or an adenoviral vector.

The coding sequence of interest may, if necessary, be operably linked to a suitable terminator or to a polyadenylation sequence. In the case of RNA, particularly siRNA, “coding sequence” refers to the sequence encoding or corresponding to the relevant RNA strand or RNA strands.

Further, the vector may comprise a DNA sequence enabling the vector to replicate in the mammalian host cell. Examples of such a sequence—particularly when the host cell is a mammalian cell—is the SV40 origin of replication.

A number of vectors suitable for expression in mammalian cells are known in the art and several of them are commercially available. Some commercially available mammalian expression vectors which may be suitable include, but are not limited to, pMC1neo (Stratagene), pXT1 (Stratagene), pSG5 (Stratagene), pcDNAI (Invitrogen), EBO-pSV2-neo (ATCC 37593), pBPV-1(8-2) (ATCC 37110), pSV2-dhfr (ATCC 37146). Preferred are all suitable gene therapeutic vectors known in the art.

In a particularly preferred embodiment of the invention the vector is a retroviral vector. Retrovinises are RNA-viruses possessing a genome that after the infection of a cell, such as a human cell, is reversely transcribed in DNA and subsequently is integrated into the genome of the host cell. Retroviruses enter their host cell by receptor-mediated endocytosis. After the endocytosis into the cell the expression of the retroviral vector may be silenced to ensure that only a single cell is infected. The integration of the viral DNA into the genome is mediated by a virus-encoded protein called integrase, wherein the integration locus is not defined. Retroviral vectors are particularly appropriate for their use in gene therapeutic methods, since their transfer by receptor-mediated endocytosis into the host cell, also kiown to those skilled in the art as “retroviral transduction” is particularly efficient. A person skilled in the art also knows how to introduce such retroviral vectors into the host cell using so called “packaging cells”.

In another particularly preferred embodiment of the invention, the vector is an adenoviral vector or a derivative thereof. Adenoviral vectors comprise both replication-capable and and replication-deficient vectors. The latter include vectors deficient in the E1 gene.

The recombinant vector is preferably introduced into the mammalian host cells by a suitable pharmaceutical carrier that allows transformation or transfection of the mammalian, in particular human cells. Preferred transformation/transfection techniques include, but are not limited to liposome-mediated transfection, virus-mediated transfection and calcium phosphate transfection.

In a preferred embodiment, the invention relates to the use of a vector system capable of producing siRNAs as defined above, wherein the nucleic acid corresponding to the siRNA is contained in at least one nucleic acid expression vector comprising a first expression cassette containing the nucleic acid corresponding to the sense-RNA-strand under the control of a first promoter and a second expression cassette containing the nucleic acid corresponding to the antisense-RNA-strand under the control of a second promoter.

In the above mentioned vector system, the vector comprises two individual promoters, wherein the first promoter controls the transcription of the sense-strand and the second promoter controls the transcription of the antisense strand (also described in Tuschl, Nature Biotechnology, Vol. 20, pp. 446-448). Finally the siRNA duplex is constituted by the hybridisation of the first and the second RNA-strand.

The term “expression cassette” is defined herein to include all components which are necessary or advantageous for the expression of a specific target polypeptide. An “expression cassette” may include, but is not limited to, the nucleic acid sequence of interest itself (e.g. encoding or corresponding to the siRNA or polypeptide of interest) and “control sequences”. These “control sequences” may include, but are not limited to, a promoter that is operatively linked to the nucleic acid sequence of interest, a ribosome binding site, translation initiation and termination signals and, optionally, a repressor gene or various activator genes. Control sequences are referred to as “homologous”, if they are naturally linked to the nucleic acid sequence of interest and referred to as “heterologous” if this is not the case. The term “operably linked” indicates that the sequences are arranged so that they finction in concert for their intended purpose, i.e. expression of the desired protein, or, in case of RNA, transcription of the desired RNA.

The promoter used in the aforementioned “expression cassettes” may be any DNA sequence which shows transcriptional activity in a host cell of choice, preferably in a mammalian host cell, particularly in a human host cell. The promoter may be derived from genes encoding proteins either homologous or heterologous to the host cell.

As a promoter in general every promoter known in the prior art can be used that allows the expression of the gene of interest under appropriate conditions in a mammalian host cell, in particular in a human host cell. Particularly promoters derived from RNA polymerase III transcription units, which normally encode the small nuclear RNAs (snRNAs) U6 or the human RNAse P RNA H1, can be used as promoters to express the therapeutic siRNAs. These particularly preferred promoters U6 and H1 RNA which are members of the type III class of Polymerase m promoters are—with the exception of the first transcribed nucleotide (+1 position)—only located upstream of the transcribed region.

In a preferred embodiment, the invention relates to the use of a vector system capable of producing siRNAs for the above identified nucleic acid sequences, wherein the sequence is contained in at least one nucleic acid expression vector comprising an expression cassette containing the sequence of the sense-RNA-strand and of the antisense-RNA-strand under the control of a promoter leading to a single-stranded RNA-molecule and wherein the single-stranded RNA-molecule is capable of forming a back-folded stem-loop-structure.

In this vector system (also described in Tuschl, Nature Biotechnology, Vol. 20, pp. 446-448), only a single RNA-strand is produced under the control of a single promoter, wherein the RNA strand comprises both the sense- and of the antisense-strand of the final double-stranded siRNA molecule. This structure leads to a back-folding of the RNA-strand by hybridisation of the complementary sense- and antisense-sequences under stem-loop formation. Finally the intracellular processing of this fold-back stem-loop-structure gives rise to siRNA.

In another preferred embodiment according to the present invention, the “nucleic acid expression vector” comprises an expression cassette containing the sequence of the sense-RNA-strand and of the antisense-RNA-strand both under the control of a single promoter leading to a single-stranded RNA-molecule. This single-stranded RNA-molecule is hereby capable to form a back-folded stem-loop-structure. These expressed “hairpin RNA-molecules” subsequently give rise to siRNAs after intracellular processing.

In a preferred embodiment of the invention the nucleic acid expression vector that gives rise to the expression of siRNAs according to the present invention is first introduced into therapeutic, non-toxic virus particles or virus-derived particles that are suitable for gene therapeutic applications and that can infect mammalian, in particular human target cells, such as packaging cells etc.

In a preferred embodiment, the first and the second RNA strand of the siRNA may have, independently from the other, a length of 19 to 25 nucleotides, more preferred of 20 to 25 nucleotides, and most preferred of 20 to 22 nucleotides.

In another preferred embodiment, the first and the second RNA strand of the siRNA may have, independently from the other, a length of 26 to 30 nucleotides, more preferred of 26 to 28 nucleotides, and most preferred of 27 nucleotides.

The present invention also relates to the use of and/or methods involving proteins, polypeptides and peptides encoded by the above defined sequences.

In another aspect, the invention relates to the use of isolated proteins or polypeptides comprising a sequence of the group selected of:
(a) a sequence as disclosed in SEQ ID NO.22, 24, 2, 4, 6, 8, 10, 12, 14, 16, 18, 20, 26, 28, 30, 32, 34, 36, 38;
(b) a sequence that exhibits a sequence identity with a sequence according to a) of at least 25% over 100 residues,
(c) or fragments of the sequences defined in (a) or (b), for the manufacture of a medicament for the inhibition of chromatin separation.

Proteins, polypeptides and peptides can be introduced into the cells by various methods known in the art. For example, amphiphilic molecules may be membrane permeable and can enter cells directly. Membrane-bound proteins or polypeptides (usually lipophilic molecules or containing transmembrane domains) may insert directly into cell membranes and can thus exert their biological function. Other ways of introduction or intracellular uptake include microinjection, lipofection, receptor-mediated endocytosis, or the use of suitable carrier-molecules, particularly carrier-peptides. Suitable carrier-peptides include or can be derived from HIV-tat, antennapedia-related peptides (penetratins), galparan (transportan), polyarginine-containing peptides or polypeptides, Pep-1, herpes simplex virus VP-22 protein. Another possible introduction method is to introduce nucleic acid vectors capable of expressing such proteins, polypeptides or peptides

Suitable methods to produce isolated polypeptides are known in the art. For example, such a method may comprise transferring the expression vector with an operably linked nucleic acid molecule encoding the polypeptide into a suitable host cell, cultivating said host cells under conditions which will permit the expression of said polypeptide or fragment thereof and, optionally, secretion of the expressed polypeptide into the culture medium. Depending on the cell-type different desired modifications, e.g. glycosylation, can be achieved.

The proteins, polypeptides and peptides may also be produced synthetically, e.g. by solid phase synthesis (Merrifield synthesis).

The polypeptides used in the invention may also include fusion polypeptides. In such fusion polypeptides another polypeptide may be fused at the N-terminus or the C-terminus of the polypeptide of interest or fragment thereof. A fusion polypeptide is produced by fusing a nucleic acid sequence (or a portion thereof) encoding another polypeptide to a nucleic acid sequence (or a portion thereof) of the present invention. Techniques for producing fusion polypeptides are known in the art and include ligating the coding sequences so that they are in frame and the expression of the fusion polypeptide is under control of the same promotor(s) and terminator.

Expression of the polypeptides of interest may also be performed using in vitro produced synthetic mRNA. Synthetic mRNA can be efficiently translated in various cell-free systems, including but not limited to, wheat germ extracts and reticulocyte extracts, as well as efficiently translated in cell based systems including, but not limited to, microinjection into frog oocytes, preferably Xenopus laevis oocytes.

Inhibition of chromatin separation using said isolated proteins or polypeptides can be achieved by different ways familiar to the person skilled in the art: Overexpression of the protein or polypeptide may lead to suppression of the endogenous protein's biological function. By introducing deletions or other mutations, or by using suitable fragments, it is possible to generate sequences encoding dominant-negative peptides or polypeptides. Such dominant-negative peptides or polypeptides can inhibit the function of the corresponding endogenous protein. For example, fragments or mutants can be generated which consist only of binding domains but are enzymatically inactive (i.e. partially lacking their biological fuiction). Such dominant-negative molecules may interfere with the biological function of the endogenous proteins or polypeptides by binding to intracellular binding partners and thus blocking activation of the endogenous molecule.

In another aspect, the invention relates to the use of an antibody which is directed against at least one polypeptide comprising a sequence as defined above for the manufacture of a medicament for the inhibition of chromatin separation.

The term “antibody” as used herein includes both polyclonal and monoclonal antibodies, as well as fragments thereof, such as Fv, Fab and F(ab)₂ fragments that are capable of binding antigen or hapten. The present invention also contemplates “humanized” hybrid antibodies wherein amino acid sequences of a non-human donor antibody exhibiting a desired antigen-specificity are combined with sequences of a human acceptor antibody.

The donor sequences will usually include at least the antigen-binding amino acid residues of the donor but may comprise other structurally and/or functionally relevant amino acid residues of the donor antibody as well. Such hybrids can be prepared by several methods well known in the art.

Specifically, said antibodies or suitable fragments thereof, particularly in humanized form, may be used as therapeutic agents in a method for treating cancer and other proliferative diseases.

The use of said antibodies may also include the therapeutical inhibition of the above identified nucleic acid molecules or their corresponding polypeptides. In particular, this use may be directed to a proliferative disease.

The antibodies or fragments may be introduced into the body by any method known in the art. Delivery of antibodies, particularly of fragments, into live cells may be performed as described for peptides, polypeptides and proteins. If the antigen is extracellular or an extracellular domain, the antibody may exert its function by binding to this domain, without need for intracellular delivery.

Antibodies can be coupled covalently to a detectable label, such as a radiolabel, enzyme label, luminescent label, fluorescent label or the like, using linker technology established for this purpose. Labeling is particularly useful for diagnostic purposes (see below) or for monitoring the distribution of the antibody within the body or a neoplastic tumor, e.g. by computed tomography, PET (positron emission tomography), or SPECT (single photon emission computed tomography).

In another embodiment, the invention relates to the use of nucleic acid molecules, peptides, polypeptides, proteins, or antibodies, as defined above, for the manufacture of a medicament for the treatment or therapy of a proliferative disease.

In a preferred embodiment, the disease is coronary restenosis or a neoplastic disease, the latter preferably selected from the group consisting of lymphoma, lung cancer, colon cancer, ovarian cancer and breast cancer (see above).

“Proliferative diseases” according to the present invention are diseases associated with excessive cell division or proliferation as for example cancer. Preferably, the proliferative disease is restenosis, particularly coronary restenois, or a neoplastic disease, the latter preferably selected from the group consisting of lymphoma, lung cancer, colon cancer, ovarian cancer and breast cancer.

Restenosis is a re-narrowing of a blood vessel due to growth of tissue at the site of angioplasty or stent implantation. Stents are tiny metal tubes to hold the previously blocked arteries open. However, restenosis still develops in many patients with implanted stents, thus necessitating second angioplasty, stent implantation or even coronary bypass surgery.

Neoplastic diseases are diseases caused by newly forming tissue or cells. In the context of the present invention, the most relevant neoplastic diseases are neoplastic tumors, particularly selected from the group consisting of lymphoma, lung cancer, colon cancer, ovarian cancer and breast cancer.

In another aspect, the invention relates to the use of an isolated nucleic acid molecule comprising a nucleic acid with a sequence selected from the group of sequences consisting of:
a) the nucleic acid sequences presented in SEQ ID NO. 21, 23, 1, 3, 5, 7, 9, 11, 13, 15, 17, 19, 25, 27, 29, 31, 33, 35, 37;
b) nucleic acid sequences encoding polypeptides that exhibit a sequence identity with the protein encoded by a nucleic acid according to a) of at least 25% over 100 residues and/or which are detectable in a computer aided search using the BLAST sequence analysis programs with an e-value of at most 10⁻⁵,
c) sequences of nucleic acid molecules which are capable of hybridizing with the nucleic acid molecules with sequences corresponding to (a) or (b) under conditions of medium or high stringency,
d) the antisense-sequence of any of the sequences as defined in (a), (b) or (c),
e) fragments of (a), (b), (c) or (d),
f) RNA sequences corresponding to any of the sequences as defined in (a), (b), (c), (d), or (e), for the manufacture of a medicament for the activation of chromatin separation.

In another aspect, the invention relates to the use of a an isolated peptide or polypeptide comprising a peptide or polypeptide with a sequence selected from the group consisting of:
(a) a sequence as disclosed in SEQ ID NO. 22, 24, 2, 4, 6, 8, 10, 12, 14, 16, 18, 20, 26, 28, 30, 32, 34, 36, 38;
(b) a sequence that exhibits a sequence identity with any of the sequences according to (a) SEQ ID NO. 21, 23, 1, 3, 5, 7, 9, 11, 13, 15, 17, 19, 25, 27, 29, 31, 33, 35, 37 of at least 25% over 100 residues.
(c) fragments of the sequences defined in (a) or (b), for the manufacture of a medicament for the activation of chromatin separation.

In another aspect, the invention relates to the use of an antibody which is directed against at least one peptide or polypeptide with a sequence as defined above for the manufacture of a medicament for the activation of chromatin separation.

Thus, another use or method involving the above identified nucleic acid sequences, peptides, polypeptides, proteins, and antibodies is directed towards the treatment of a disease in which chromatin separation during cell division is abnormal, deficient or negatively affected.

Diseases with abnormal, deficient or negatively affected chromatin separation during cell division may be characterized by increased apoptosis and developmental disorders, in particular growth retardation, or slowed wound healing.

Therefore, a preferred embodiment of the present invention relates to a use or method of the treatment of a disease, wherein the disease is characterized by increased apoptosis, growth retardation, or slowed wound healing.

“Activation of chromatin separation” includes both initiation and stimulation of chromatin separation.

The use may include, but is not limited to, the use of said nucleic acid molecules and their corresponding polypeptides for direct or indirect activation of the expression of said target genes and/or for activation of the function of said target genes. In particular, the use may include the replacement for or the complementation of a lack of finction or activity of an endogenous gene involved in chromatin separation during cell division.

Expression of RNA or polypeptides may be achieved by introduction of genomic DNA or cDNA containing suitable promoters, preferably constitutive or homologous promoters. Alternatively, any suitable nucleic acid expression vector can be used (see also above). The encoded protein or polypeptide may be full-length or a fragment or peptide with a similar biological finction in chromatin separation, particularly with the capability to activate chromatin separation.

All gene therapy techniques kIown in the art can be used to introduce the sequences into cells or tissues of a subject suffering from a disease negatively affecting chromatin separation during cell division. Particularly useful for introduction of the above identified sequences are viral vectors, e.g. retroviral or adenoviral vectors, lipofection and electroporation.

The proteins, polypeptides or peptides may also be generated by any known in vivo or in vitro method and introduced directly into the cells (see above).

It is known that suitable antibodies can be used to activate the biological function of target proteins they bind to. Activation may occur by inducing conformational changes upon binding to the target protein. Another possibility is that the antibody binds two or more target proteins and brings them into sufficiently close physical proximity to induce interaction of the target proteins. The latter mode of activation is particularly known for membrane-bound dimeric receptors.

With respect to the specific embodiments relating to the used nucleic acids, peptides, polypeptides, proteins, and antibodies the same applies as defined above for the other uses of the invention.

In another embodiment, the invention relates to a medicament containing an isolated nucleic acid molecule, peptide, polypeptide, or antibody selected from the group consisting of
a) nucleic acid molecules or nucleic acid expression vectors as defined above,
b) a peptide br polypeptide comprising a sequence as defined above,
c) an antibody directed against at least one peptide or polypeptide according to (b).

Preferably this isolated nucleic acid molecule is an RNA molecule and preferably is double-stranded. Particularly the isolated nucleic acid molecule is an siRNA molecule according to the present invention.

The medicaments may be used or applied in methods for the therapy of any kind of proliferative disease, such as cancer, preferably for the therapy of diseases in which chromatin separation during cell division plays a role, particularly for the therapy of a lymphoma, lung cancer, colon cancer, ovarian cancer or breast cancer.

The medicaments may also be used or applied in methods for the therapy of any kind of disease associated with abnormal or deficient chromatin separation during cell division, particularly diseases characterized by increased apoptosis, developmental disorders or abnormalities (particularly growth retardation) and slowed wound healing.

The following considerations for medicaments and their administration apply also to the medicaments of the invention as to the above disclosed uses.

The medicament preferably comprises additionally a suitable pharmaceutically acceptable carrier, preferably virus-particles or virus-derived particles that may harbour the viral vectors, transfection solutions comprising liposomes, particularly cationic liposomes, calcium phosphate etc. Preferably a carrier is used, which is capable of increasing the efficacy of the expression vector or virus particles containing the expression vector to enter the mammalian target cells. The medicament may additionally comprise other carrier substances, preferably starch, lactose, fats, stearin acid, alcohol, physiological NaCl-solutions or fer additives, in particular stabilizers, preservatives, dyes and flavourings.

The medicaments may also comprise other suitable substances. For example, RNA or siRNA containing medicaments may contain substances which stabilize double-stranded RNA molecule and/or which enable the double-stranded RNA molecule or DNA expression vector to be transfected or to be injected into the human or animal cell.

Administration can be carried out by known methods, wherein a nucleic acid is introduced into a desired cell in vitro or in vivo. For therapeutic applications, the medicament may be in form of a solution, in particular an injectable solution, a cream, ointment, tablet, suspension, granulate or the like. The medicament may be administered in any suitable way, in particular by injection, by oral, nasal, rectal application. The medicament may particularly be administered parenteral, that means without entering the digestion apparatus, for example by subcutaneous injection. The medicament may also be injected intravenously in the form of solutions for infusions or injections. Other suitable administration forms may be direct administrations on the skin in the form of creams, ointments, sprays and other transdermal therapeutic substances or in the form of inhalative substances, such as nose sprays, aerosoles or in the form of microcapsules or implantates.

The optimal administration form and/or administration dosis for a medicament either comprising double-stranded RNA molecules with the above sequences or comprising nucleic acid vectors capable to express such double-stranded RNA molecules depend on the type and the progression of the disease to be treated.

Preferably, the activator or inhibitor is administered in pharmaceutically effective amount. As used herein, a “pharmaceutically effective amount” of an activator or inhibitor is an amount effective to achieve the desired physiological result, either in cells treated in vitro or in a subject treated in vivo. Specifically, a pharmaceutically effective amount is an amount sufficient to positively influence, for some period of time, one or more clinically defined pathological effects associated with proliferative diseases or diseases associated with abnormal or deficient chromatin separation during cell division The pharmaceutically effective amount may vary depending on the specific activator or inhibitor selected, and is also dependent on a variety of factors and conditions related to the subject to be treated and the severity of the disease. For example, if the activator or inhibitor is to be administered in vivo, factors such as age, weight, sex, and general health of the patient as well as dose response curves and toxicity data obtained in pre-clinical animl tests would be among the factors to be considered. If the activator or inhibitor is to be contacted with cells in vitro, one would also design a variety of pre-clinical in vitro studies to asses parameters like uptake, half-life, dose, toxicity etc. The determination of a pharmaceutically effective amount for a given agent (activator or inhibitor) is well within the ability of those skilled in the art. Preferably, the activator or inhibitor is present in a concentration of 0.1 to 50% per weight of the pharmaceutical composition, more preferably 10 to 30%.

An inhibitor, activator, or drug according to the present invention may also be a “small molecule”. Small molecules are molecules which are not proteins, peptides antibodies or nucleic acids, and which exhibit a molecular weight of less than 5000 Da, preferably less than 2000 Da, more preferably less than 2000 Da, most preferably less than 500 Da Such small molecules may be identified in high throughput procedures/screening assays starting from libraries. Such methods are known in the art Suitable small molecules can also be designed or firther modified by methods known as combinatorial chemistry.

The genes/proteins that are provided by the current application and that possess one of the sequences as defined in (a) to (f), can be used in a high-throughput or other screen for new agents that inhibit or activate the mitotic process of chromatin separation. Particularly inhibitors of chromatin separation identified by such a screen may be used as medicaments for the therapy of proliferative diseases, particularly for the therapy of a disease in which chromatin separation during cell division plays a role.

In another aspect, the present invention relates to the use of an isolated nucleic acid molecule comprising a sequence as defined above or the use of a ligand binding specifically at least one polypeptide comprising a sequence as defined above for the in vitro diagnosis of a proliferative disease or a disease associated with abnormal chromatin separation.

In a preferred embodiment, diagnosis relates to proliferative diseases as defined above. In another preferred embodiment, diagnosis relates to diseases associated with abnormal, deficient or negatively affected chromatin separation during cell division, as they are described above. Diseases with “abnormal” chromatin separation include diseases in which chromatin separation is deficient or negatively affected.

In a proliferative disease, expression of endogenous genes corresponding to the above identified sequences may be increased.

In a disease in which chromatin separation is abnormal, deficient or negatively affected, expression of the corresponding endogenous genes may be lowered. Furthermore, the corresponding endogenous gene may be mutated, rendering the corresponding protein less active or non-functional.

The diagnostic use of the above identified nucleic acid molecules and probes may include, but is not limited to the quantitative detection of expression of said target genes in biological probes (preferably, but not limited to tissue samples, cell extracts, body fluids, etc.), particularly by quantitative hybridization to the endogenous nucleic acid molecules comprising the above-characterized nucleic acid sequences (particularly cDNA, RNA)

Expression of the endogenous genes or their corresponding proteins can be analyzed in vitro in tissue samples, body fluids, and tissue and cell extracts. Expression analyis can be performed by any method known in the art, such as RNA in situ hybridization, PCR (including quantitative RT-PCR), and various serological or immunological assays which include, but are not limited to, precipitation, passive agglutination, enzyme-linked immunosorbent antibody (ELISA) technique and radioinrnunoassay techniques.

The diagnostic use may also include the detection of mutations in endogenous genes corresponding to the above identified nucleic acid sequences.

Suitable nucleic acid probes may be synthesized by use of DNA synthesizers according to standard procedures or, preferably for long sequences, by use of PCR technology with a selected template sequence and selected primers. The probes may be labeled with any suitable label known to those skilled in the art, including radioactive and non-radioactive labels. Typical radioactive labels include ³²P, ¹²⁵I, ³⁵S, or the like. A probe labeled with a radioactive isotope can be constructed from a DNA template by a conventional nick translation reaction using a DNase and DNA polymerase. Non-radioactive labels include, for example, ligands such as biotin or thyroxin, or various luminescent or fluorescent compounds. The probe may also be labeled at both ends with different types of labels, for example with an isotopic label at one end and a biotin label at the other end. The labeled probe and sample can then be combined in a hybridization buffer solution and held at an appropriate temperature until annealing occurs. Such nucleic acid probes may also be used for other than diagnostic purposes, e.g. for the identification of further homologs or orthologs.

“Ligands” binding specifically to said polypeptides are known in the art. Such ligands include proteins or polypeptides, for example intracellular binding partners, antibodies, molecular affinity bodies, and small molecules. Specifically binding ligands can be identified by standard screening assays known in the art (see also below), for example by yeast two-hybrid screens and afity chromatography. A specifically binding ligand does not need to exert another function such as inhibiting or activating the molecule with which it interacts.

In a preferred embodiment, the ligand is an antibody binding specifically at least one polypeptide comprising a sequence as defined above.

“Specific binding” according to the present invention means that the polypeptide to be identified (the target polypeptide) is bound with higher affinity than any other polypeptides present in the sample. Preferred is at least 3 times higher affinity, more preferred at least 10 times higher affinity, and most preferred at least 50 times higher affinity. Non-specific binding (“cross-reactivity”) may be tolerable if the target polypeptide can be identified unequivocally, e.g. by its size on a Western blot.

Preferably the specifically binding ligands can be labeled, e.g. with fluorescent labels, enzymes, molecular tags (e.g. GST, myc-tag or the like), radioactive isotopes, or with labeled substances, e.g. labeled secondary antibodies. For MRI (magnetic resonance imaging), the ligands may be chelated with gadolinium, superparamagnetic iron oxide or lanthanides. For PET (positron emission tomography) or SPECT (single photon emission computed tomography) commonly used isotopes include ¹¹C, ¹⁸F, ¹⁵O, ¹³N, ⁸⁶Y, ⁹⁰Y, and ¹⁶Co.

In another aspect, the present invention relates to a diagnostic kit containing an isolated nucleic acid molecule as defined above and/or a ligand which is directed against at least one polypeptide as defined above for the in vitro diagnosis of a proliferative disease or a disease associated with abnormal chromatin separation.

Diagnostic kits may comprise suitable isolated nucleic acid or amino acid sequences of the above identified genes or gene products, labelled or unlabelled, and/or specifically binding ligands (e.g. antibodies) thereto and auxiliary reagents as appropriate and known in the art The assays may be liquid phase assays as well as solid phase assays (i.e. with one or more reagents immobilized on a support). The diagnostic kits may also include ligands directed towards other molecules indicative of the disease to be diagnosed.

In another aspect, the invention relates to the use of an isolated nucleic acid molecule or a nucleic acid expression vectors as defined above or of an antibody which is directed against at least one polypeptide comprising a sequence as defined above, in a screening assay for the identification and characterization of drugs that inhibit or activate chromatin separation.

In another aspect, the invention relates to the use of a peptide, polypeptide or protein with a sequence as defined above in a screening assay for interacting drugs, that inhibit or activate chromatin separation during cell division. Such interacting molecules may-also be used as ligands for diagnosis as described above.

“Screening assay” according to the present invention relates to assays which allow to identify substances, particularly potential drugs, that inhibit or activate chromatin separation by screening libraries of substances. “Screening assay” according to the present invention also relates to assays to screen libraries for substances capable of binding to the nucleic acids, polypeptides, peptides or antibodies defined above. Suitable libraries may, for example, include small molecules, peptides, polypeptides or antibodies.

The invention relates to assays for identification as well as to assays for characterization of substances that inhibit or activate chromatin separation or bind to said nucleic acids, polypeptides, peptides or antibodies. Particularly, the invention relates to screening assays for drugs. Such drugs may be identified and characterized from libraries of unspecified compounds as well as libraries of drugs which are already known for treatment of other diseases. For such known drugs also potential side-effects and therapeutically applicable doses are known.

Suitable drugs include “interacting drugs”, i.e. drugs that bind to the polypeptides or nucleic acids identified above. Such interacting drugs may either inhibit or activate the molecule they are bound to. Examples for interacting substances are peptide nucleic acids comprising sequences identified above, antisense RNAs, siRNAs, ribozymes, aptamers, antibodies and molecular affinity bodies (CatchMabs, Netherlands). Such drugs may be used according to any aspect of the present invention, including use for the manufacture of medicaments and methods of treatment of proliferative diseases. It is known that such interacting drugs can also be labeled and used as ligands for diagnosis of a disease associated with chromatin separation during cell division.

Suitable screening assays are known in the art. For example, in a preferred embodiment of the invention the screening method for the identification and characterization of an inhibitor or an activator molecule that inhibits or activates chromatin separation during cell divison comprises the following steps:
a) transformation of a nucleic acid molecule or a nucleic acid expression vector as defined above into a host cell or host organism,
b) cultivation of the host cell or host organism obtained in step a) under conditions that allow the overexpression of the polypeptide or RNA encoded by or corresponding to the nucleic acid of step (a) either in the presence or in the absence of at least one candidate for an inhibitor- or activator-molecule, and
c) analysis of the chromatin separation in the cultivated cell or organism and thereby identification of an inhibitor or activator of chromatin separation.

The term “expression vector” as used herein does not only relate to RNA or siRNA expressing vectors, but also to vectors expressing peptides, polypeptides or proteins.

The transfer of the expression vector into the host cell or host organism hereby may be performed by all known transformation or transfection techniques, including, but not limited to calcium phosphate transformation, lipofection, microinjection. Host cell/host organisms may be all suitable cells or organisms that allow detection of an impaired cell division, preferably of an impaired chromatin separation. A particularly preferred host organism is C. elegans, since its translucent body allows an easy detection of failures during cell division, including chromatin separation. Human cells, preferably mammalian, more preferably human cells, in particular human cell lines are also preferred host cells. The expression vector may be any known vector that is suitable to allow the expression of the nucleic acid sequence as defined above. Preferred expression vectors possess expression cassettes comprising a promoter that allows an overexpression of the RNA, peptide or polypeptide as defined above.

After the transfer of the expression vector into the host cell/host organism one part of the host cells.or host organisms are cultured in the presence of at least one candidate of an inhibitor- or activator-molecule and under culture conditions that allow the expression, preferably the overexpression of the RNA, peptide or polypeptide as defined above. The other part of the transfected host cells are cultured under the same culture conditions, but in the absence of the candidate of an inhibitor- or activator-molecule.

Finally, after an appropriate incubation time/culture period the proliferation state and/or cell divisions for host cells or host organisms that had been cultured in the presence or in the absence of the at least one candidate for an inhibitor or an activator molecule are detected or preferably quantified. This detection or quantification step is preferably done by time lapse fluorescence or DIC microscopy, particularly in those cases when the host organism is C. elegans or another mostly translucent organism that is available to be analysed by time lapse fluorescence or DIC microscopy. The detection/quantification step may also be done by any other technique known to the state of the art that is suitable to analyse the proliferation state or the extent of cell division, preferably all kinds of microscopic techniques.

In another preferred embodiment, the screening method for the identification and characterization of an interacting molecule that inhibits or activates chromatin separation during cell divison from a library of test substances comprises the following steps:
a) recombinantly expressing a polypeptide encoded by a nucleic acid molecule sequence as defined in above in a host cell,
b) isolating and optionally purifying the recombinantly expressed polypeptide of step (a),
c) optionally labelling of the test substances and/or labelling of the recombinantly expressed polypeptide,
d) immobilizing the recombinantly expressed polypeptide to a solid phase,
e) contacting of at least one test substance with the immobilized polypeptide,
f) optionally one or more washing steps,
g) detecting the binding of the at least one test substance to the immobilized polypeptid at the solid phase, and
h) performing a functional assay for inhibition or activation of chromatin separation.

Step a) includes the recombinant expression of the above identified polypeptide or of its derivative from a suitable expression system, in particular from cell-free translation, bacterial expression, or baculuvirus-based expression in insect cells.

Step b) comprises the isolation and optionally the subsequent purification of said recombinantly expressed polypeptides with appropriate biochemical techniques that are familiar to a person skilled in the art.

Alternatively, these screening assays may also include the expression of derivatives of the above identified polypeptides which comprises the expression of said polypeptides as a fusion protein or as a modified protein, in particular as a protein bearing a “tag”-sequence. These “tag”-sequences consist of short nucleotide sequences that are ligated ‘in frame’ either to the N- or to the C-terminal end of the coding region of said target gene. Commonly used tags to label recombinantly expressed genes are the poly-Histidine-tag which encodes a homopolypeptide consisting merely of histidines, particularly six or more histidines, GST (glutathion S-transferase), c-myc, FLAG®, MBP (maltose binding protein), and GFP. In this context the term “polypeptide” does not merely comprise polypeptides with the nucleic acid sequences of SEQ ID No. 1 to 37, their naturally occuring homologs, preferably orthologs, more preferably human orthologs, but also derivatives of these polypeptides, in particular fusion proteins or polypeptides comprising a tag-sequence.

These polypeptides, particularly those labelled by an appropriate tag-sequence (for instance a His-tag or GST-tag), may be purified by standard affinity chromatography protocols, in particular by using chromatography resins linked to anti-His-tag-antibodies or to anti-GST-antibodies which are both commercially available. Alternatively, His-tagged molecules may be purified by metal chelate affinity chromatography using Ni-ions. Alternatively to the use of ‘label-specific’ antibodies the purification may also involve the use of antibodies against said polypeptides. Screening assays that involve a purification step of the recombinantly expressed target genes as described above (step 2) are-preferred embodiments of this aspect of the invention.

In an—optional—step c) the compounds tested for interaction maybe labelled by incorporation of radioactive isotopes or by reaction with luminescent or fluorescent compounds. Alternatively or additionally also the recombinantly expressed polypeptide maybe labelled.

In step d) the recombinantly expressed polypeptide is immobilized to a solid phase, particularly (but not limited) to a chromatography resin. The coupling to the solid phase is thereby preferably established by the generation of covalent bonds.

In step e) a candidate chemical compound that might be a potential interaction partner of the said recombinant polypeptide or a complex variety thereof particularly a drug library) is brought into contact with the immobilized polypeptide.

In an—optional—step f) one or several washing steps may be performed. As a result just compounds that strongly interact with the immobilized polypeptide remain bound to the solid (immobilized) phase.

In step g) the interaction between the polypeptide and the specific compound is detected, in particular by monitoring the amount of label remaining associated with the solid phase over background levels.

Such interacting molecules may be used without flnctional characterization for diagnostic purposes as described above.

In step h) the interacting molecule is further analyzed for inhibition or activation of chromatin separation. Such analysis or functional assay can be performed according to any assay system known in the art. A suitable assay may include the cultivation of a host cell or host organism in the presence (test condition) or absence (control condition) of the interacting molecule, and comparison of chromatin separation under test and control conditions.

In another aspect, the invention relates to a method for the preparation of a pharmaceutical composition wherein an inhibitor or activator of chromatin separation is identified according to any of the screening methods described above, synthesized in adequate amounts and formulated into a pharmaceutical composition.

Suitable methods to synthesize the inhibitor or activator molecules are known in the art. For example, peptides or polypeptides can be synthesized by recombinant expression (see also above), antibodies can be obtained from hybridoma cell lines or immunized animals. Small molecules can be synthesized according to any known organic synthesis methods.

Adequate amounts relate to pharmaceutically effective amounts.

Similarly, said inhibitor or activator may be provided by any of the screening methods described above and formulated into a pharmaceutical composition.

### BRIEF DESCRIPTION OF THE DRAWINGS

FIG. 1 shows DIC microscopy images taken from time-lapse recording of the first two rounds of cell division in C. elegans F1 progeny from F0 an parent treated with dsRNA (RNAi) directed against F55C5.4.

FIG. 2 shows an amino acid sequence alignment of F55C5.4 and the corresponding human ortholog.

FIG. 3 shows DIC microscopy images taken from time-lapse recording of the first two rounds of cell division in C. elegans F1 progeny from F0 a parent treated with dsRNA (RNAi) directed against C33D12.3.

FIG. 4 shows an amino acid sequence alignment of C33D12.3 and its corresponding homologs

FIG. 5 shows DIC microscopy images taken from time-lapse recording of the first two rounds of cell division in C. elegans F1 progeny from F0 a parent treated with dsRNA (RNAi) directed against K12D12.2.

FIG. 6 shows an amino acid sequence alignment of K12D12.2 and its corresponding orthologs

FIG. 7 shows DIC microscopy images taken from time-lapse recording of the first two rounds of cell division in C. elegans F1 progeny from F0 a parent treated with dsRNA (RNAi) directed against Y48ElB.12.

FIG. 8 shows an amino acid sequence alignment of Y48E1B.12 and its corresponding human homolog

FIG. 9 shows DIC microscopy images taken from time-lapse recording of the first two rounds of cell division in C. elegans F1 progeny from F0 a parent treated with dsRNA (RNAi) directed against Y110A7A.1a. FIG. 10 shows an amino acid sequence alignment of Y110A7A.1a and its corresponding human ortholog

FIG. 11 shows DIC microscopy images taken from time-lapse recording of the first two rounds of cell division in C. elegans F1 progeny from F0 a parent treated with dsRNA (RNAi) directed against W02D9.2.

FIG. 12 shows an amino acid sequence alignment of W02D9.2 and its corresponding human homolog

FIG. 13 shows DIC microscopy images taken from time-lapse recording of the first two rounds of cell division in wild type untreated C. elegans.

FIG. 14 shows the remaining mRNA levels after RNAi treatment of HeLa cells. RNAi treatment of HeLa cells wiih siRNAs directed against AAH12469, XP_{—}166201.1, NP_{—}055032.1, NP_{—}060230.2, XP_{—}027054 and XP_{—}058073.3, the Human orthologs of C. elegans genes W02D9.2, Y110A7A.1a, C33D12.3, F55C5.4,

Y48E1B.12 and K12D12.2 respectively, results in the specific reduction to mRNA levels below 10% compared to control treated samples. mRNA, remaining mRNA levels (% of negative control treated sample); pos. ctrl., positive control; neg. ctrl., negative control.

FIG. 15 shows the effect of RNAi treatment on cell proliferation, apoptosis rate, and mitosis in HeLa cells. For graphical presentation, proliferation, apoptosis rate, and MI of untreated cells were set to 100. prolif., proliferation; apopt., apoptosis; MI, mitotic index; %, percent compared to untreated control; scr. ctrl., scrambled control; untrtd., untreated;

The following examples illustrate the present invention without, however, limiting the same thereto.

### EXAMPLE 1

Generation of dsRNA Molecules for RNAi Experiments

First, oligonucleotide primer pair sequences were selected to amplify portions of the gene of interest's coding region using standard PCR techniques. Primer pairs were chosen to yield PCR products containing at least 500 bases of coding sequence, or a maximum of coding bases for genes smaller than 500 bases. In order to permit the subsequent use of the PCR product as a template for in vitro RNA transcription reactions from both DNA strands, the T7 polymerase promoter sequence “TAATACGACTCACTATAGG” was added to the 5′ end of forward primers, and the T3 polymerase promoter sequence “AATTAACCCTCACTAAAGG” was added to the 5′ end of reverse primers. The synthesis of oligonucleotide primers was completed by a commercial supplier (Sigma-Genosys, UK or MWG-Biotech, Germany).

PCR reactions were performed in a volume of 50 μl, with Taq polymnerase using 0.8 μM primers and approximately 0.1 μg of wild-type (N2 strain) genomic DNA template. The PCR products were EtOH precipitated, washed with 70% EtOH and resuspended in 7.0 μl TE. 1.0 μl of the PCR reaction was pipetted into each of two fresh tubes for 5 μl transcription reactions using T3 and T7 RNA polymerases. The separate T3 and T7 transcription reactions were performed according to the manufacturer's instructions (Ambion, Megascript kit), each diluted to 50 μl with RNase-free water and then combined.

The mixed RNA was purified using RNeasy kits according to the manufacturer's instructions (Qiagen), and eluted into a total of 130 μl of RNase-free H₂0. 50 μl of this was mixed with 10 μl 6×injection buffer (40 mM KPO4 pH 7.5, 6 mM potassium citrate, pH 7.5, 4% PEG 6000). The RNA was annealed by heating at 68° C. for 10 min, and at 37° C. for 30 min. Concentration of the final dsRNAs were measured to be in the range of 0.1-0.3 μg/μl. The products of the PCR reaction, of the T3 and T7 transcription reactions, as well as the dsRNA species were run on 1% agarose gels to be examined for quality control purposes. Success of double stranding. was assessed by scoring shift in gel mobility with respect to single stranded RNA, when run on non-denaturing gels.

### EXAMPLE 2

Injections of dsRNA and Phenotypic Assays

dsRNAs were injected bilaterally into the syncytial portion of both gonads of wild-type (N2 strain) young adult hermaphrodites, and the animals incubated at 20° C. for 24 hrs. Embryos were then dissected out from the injected animals and analyzed by time-lapse differential interference contrast videomicroscopy for potential defects in cell division processes, capturing 1 image every 5 seconds, as previously described (Gönczy et al., Dissection of cell division processes in the one cell stage Caenorhabditis elegans embryo by mutational analysis. J Cell Biol, 144, 927-946 (1999)). For each experiment, embryos from at least 3 different injected worms were filmed in this manner, from shortly after fertilization until the four cell stage. Embryos from 2 additional injected worms were recorded for shorter periods, covering the 2 cell and the 4 cell stage, respectively, thus yielding documentation for at least 5 injected worms in each experiment.

In some cases, embryos exhibited acute sensitivity to osmotic changes, as evidenced by their loss of structural integrity during the dissection of the injected animals. In order to overcome this limitation, injected animals were not dissected, but rather, anaesthetized for 10 min in M9 medium containing 0.1% tricaine and 0.01% tetramisole, and mounted intact on an agarose pad to observe the F1 embryogenesis in utero (Kirby et al., Dev. Biol. 142, 203-215 (1990)). The resolution achieved by viewing through the body wall does not equal that achieved by observing dissected embryos, and only limited phenotypic analysis was conducted in these cases.

Three injected worms were also transferred to 3 fresh plates 24 hrs after injection of the dsRNA, and left at 18° C. Three days later, the plates were checked with a stereomicroscope (20-40×total magnification) for the presence of F1 larvae (L2's-L4's), as well as their developmental stage. Three days after that, the plates were inspected again for the presence of F1 adults, as well as their overall body morphology and the presence of F2 progeny.

### EXAMPLE 3

Characterization of the C. elegans Gene F55C5.4

dsRNA was designed and used to specifically silence the expression of the C. elegans gene by RNAi, thereby testing its finctional involvement in the first 2 rounds of embryonic cell division in this metazoan species. The dsRNA was synthesized in vitro from PCR-amplified wild type genomic DNA fragments of the F55C5.4 gene. For PCR, the following primer pair was used: “TAATACGACTCACTATAGGTGGTGTTGGAAATGGACTGA” with “AATTAACCCTCACTAAAGGAAGAAGCCAGAATTCGCTGA” as forward and reverse primers, respectively. The dsRNA was purified, and injected into adult hermaphrodite worms. The phenotypic consequences of the RNAi treatment were documented 24 hours later in the F1 progeny of injected worms, using time-lapse differential interference contrast (DIC) microscopy. Embryo recordings started ˜20 minutes after ferilization, while the female pronucleus is completing its meiotic divisions, until the 4 cell stage, ˜30 minutes later.

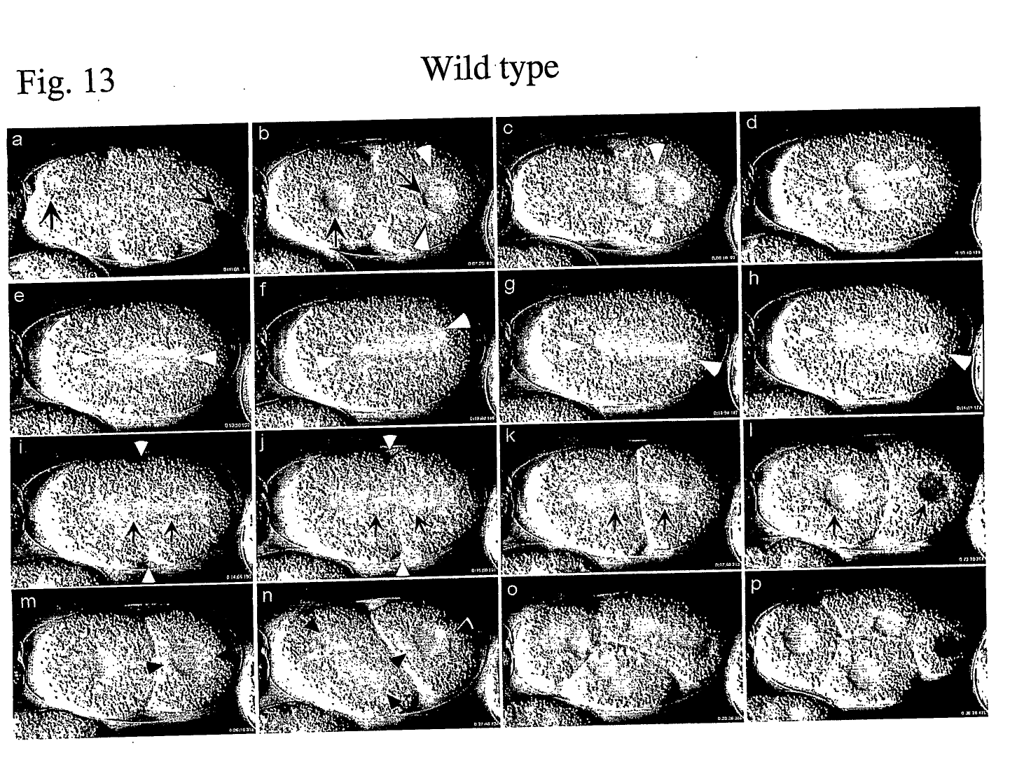

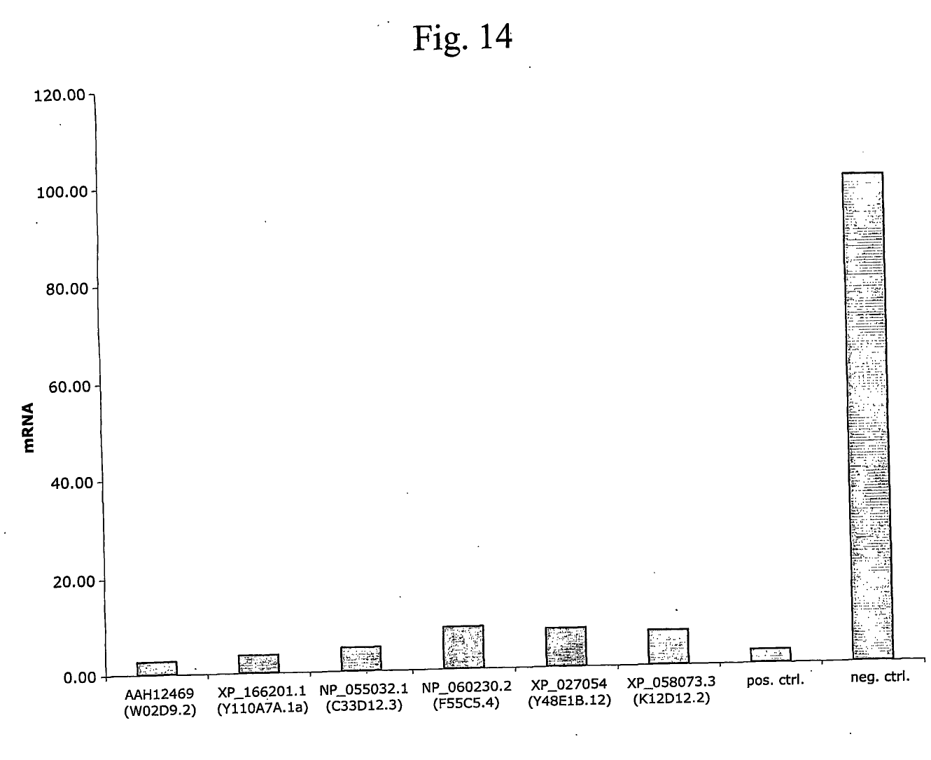

Control worms were either not injected, or injected with irrelevant dsRNA. Irrelevant dsRNA was made of the same nucleotide composition as the experimental dsRNA, but the nucleotides were in random order. In the F1 progeny of such control worms the cellular events of the first two rounds of embryonic cell division were found to exhibit very limited variability, as observed by DIC microscopy. All processes that were examined and scored for the possibility of phenotypic deviations are listed and illustrated in FIG. 13. Briefly, the antero-posterior polarity of the embryo is initially deterrnined by the position of the male pronucleus at the cortex, shortly after entry into the egg. This is accompanied by a clear, coordinated flow of yolk granules through the central portion of the cytoplasm along the embryo's longitudinal axis towards the male pronucleus, and a concomitant series of cortical waves or ruffles progressing towards the anterior of the embryo. Shortly thereafter, the male and female pronuclei undergo highly patterned migrations resulting in their meeting within the posterior half of the embryo, followed by a centration and rotation of the pronuclear pair and associated centrosomes to set up the future mitotic spindle along the embryo's longitudinal axis. After synchronous breakdown of the pronuclear envelopes, the clearly bipolar mitotic spindle is initially short, but then elongates. These movements are accompanied by a slight posterior displacement of the posterior spindle pole, while the anterior spindle pole remains approximately stationary. This then results in an asymmetric positioning of the spindle during anaphase and telophase, thereby yielding an asymmetric placement of the cytokinetic furrow, and generating unequally-sized daughter cells: a smaller posterior P1 blastomere, and larger anterior AB blastomere. While the AB nucleus then migrates directly to the center of the AB cell, the P1 nucleus typically migrates further towards the posterior of that cell, before undergoing a pronounced 90° rotation while re-migrating to the anterior P1 cortex with one of its duplicated centrosomes leading. This insures that the P1 blastomere then divides along the embryo's longitudinal axis, perpendicular to that of the AB blastomere. These two divisions occur asynchronously, with P1 lagging 2-3 minutes behind AB.

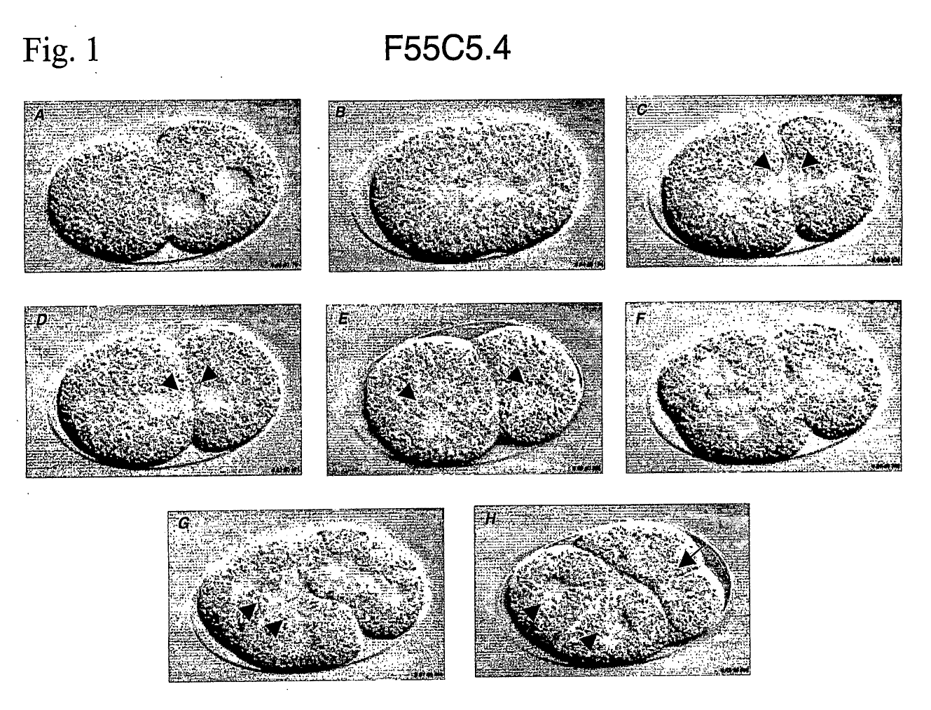

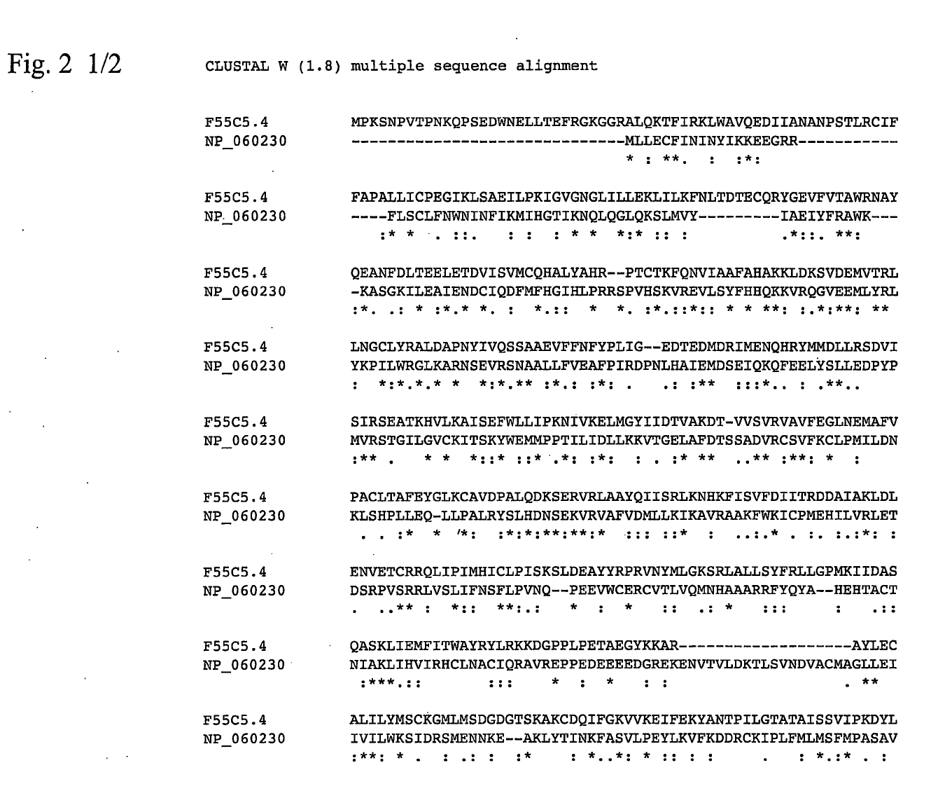

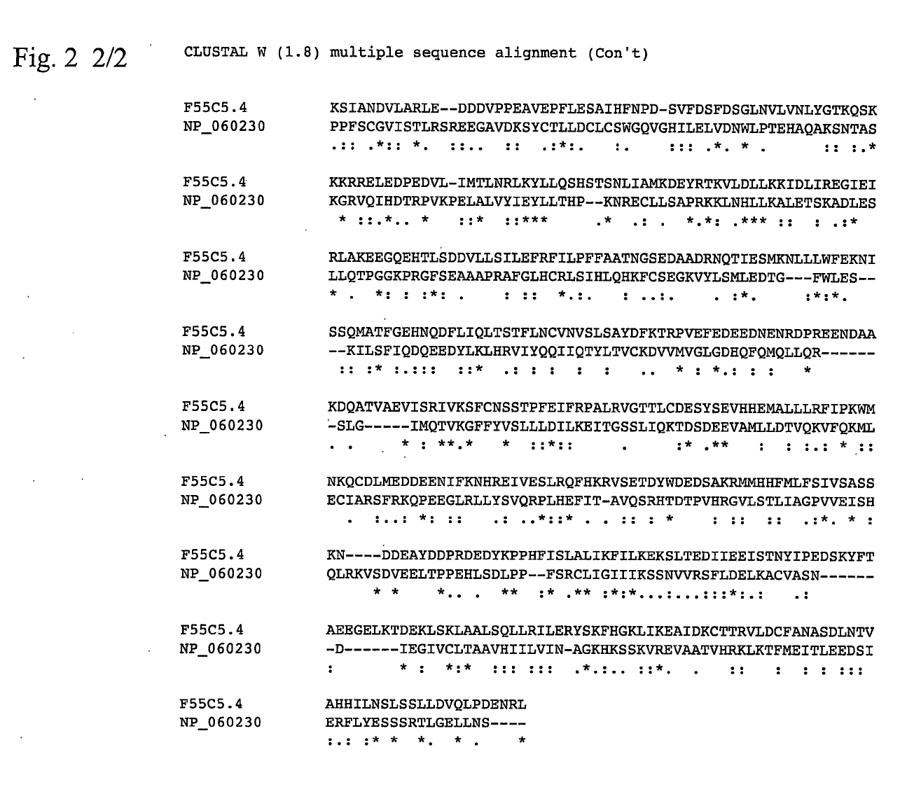

In the F1 embryos of worms injected with dsRNA, the following highly reproducible phenotypes are observed (FIG. 1). Pronuclear meeting and spindle formation occur normally (FIG. 1 A, B). However, the chromatin subsequently fails to segregate, resulting in a chromatin bridge between the newly forming nuclei (FIG. 1 C,D, arrow heads), both of which are deformed and poorly visible (FIG. 1E, arrow heads). The same pattern emerges after the second round of division (FIG. 1 F, G, H). Chromatin bridges can lead to failure of cytokinesis completion (H, long arrow).

All observed phenotypes indicate a requirement for F55C5.4 gene function in chromatin separation during mitosis. Since this function is essential to cell division throughout metazoans, this gene and any homologs and derivatives thereof represent excellent tools for use in the development of a wide range of therapeutics including anti-proliferative agents. Analysis of the F55C5.4 gene sequence reveals a clear ortholog in human (GenBank Accession No. NM₁₃ 060230) which has had only a putative function in embryogenesis and morphogenesis ascribed to it until now. There has been no information linking the ortholog to cell division or to chromatin separation during cell division. Based on the extremely high sequence conservation at the protein level, it can be concluded that this gene most likely encodes a protein with equivalent functions in chromatin separation in humans.

### EXAMPLE 4

Characterization of the C. Elegans Gene C33D12.3

dsRNA was designed and used to specifically silence the expression of the C. elegans gene by RNAi, thereby testing its functional involvement in the first 2 rounds of embryonic cell division in this metazoan species. dsRNA was synthesized in vitro from PCR-amplified wild type genomic DNA fragments of the C33D12.3 gene. For PCR, the following primer pair was used: “TAATACGACTCACTATAGGGTACGTACCCGGTTTGTTGC” with “AATTAACCCTCACTAAAGGAAAGCAAATTTCAGGAGCGA” as forward and reverse primers, respectively. The dsRNA was purified, and injected into adult hermaphrodite worms. The phenotypic consequences of the RNAi treatment were documented 24 hours later in the F1 progeny of injected worms, using time-lapse differential interference contrast (DIC) microscopy. Embryo recordings started ˜20 minutes after ferilization, while the female pronucleus is completing its meiotic divisions, until the 4 cell stage, ˜30 minutes later.

In the F1 progeny of control worms that were either not injected, or injected with irrelevant dsRNA, the cellular events of the first two rounds of embryonic cell division were found to exhibit very limited variability, as observed by DIC microscopy and described in Example 3.

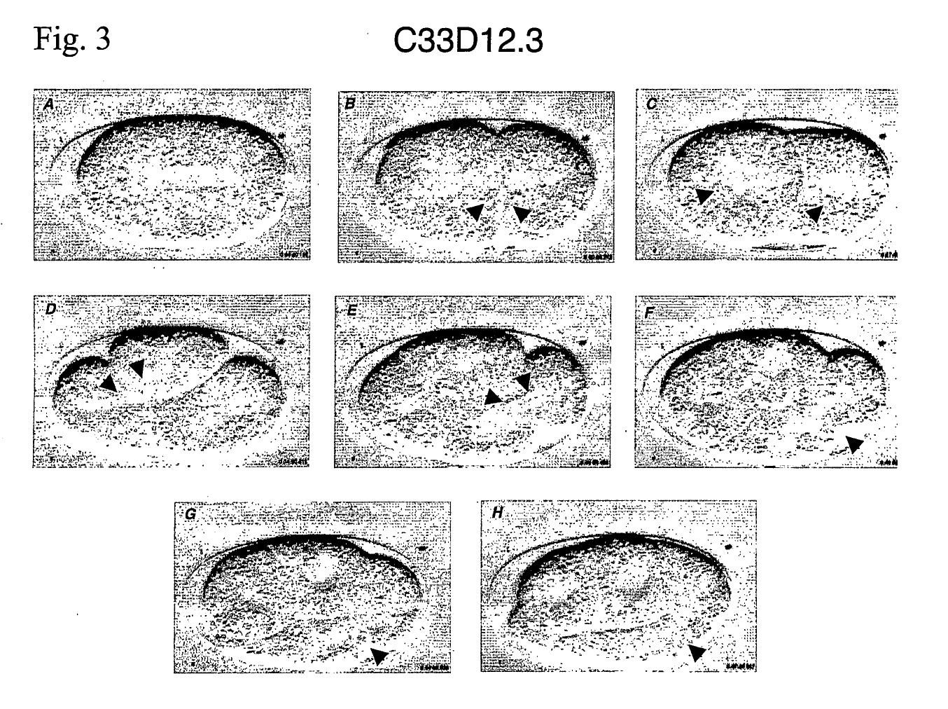

In the F1 embryos of worms injected with dsRNA, the following highly reproducible phenotypes are observed (FIG. 3). After cell division, the re-forming nuclei stay attached to each other via a chromatin bridge, resulting in a tear-shaped appearance (FIG. 3 B to E). This can result in a failure of cytolidnesis completion (FIG. 3 F to H, arrows).

All observed phenotypes indicate a requirement for C33D12.3 gene function in chromatin separation during mitosis. Since this function is essential to cell division throughout metazoans, this gene and any homologs and derivatives thereof represent excellent tools for use in the development of a wide range of therapeutics including anti-proliferative agents. Analysis of the C33D12.3 gene sequence has not revealed clear orthologsin other species. A stretch of amino acids 150 to 289 contains homology to human, mouse, and rat potassium channel, subfamily K (GenBank accession No. NP_{—}055032, XP_{—}109361, NP_{—}742039). There is low homology to a Drosophila gene product from 50-202 aa (GenBank accession No. AAF47456). There is homology to many other C. elegans proteins that have been termed potassium channel proteins. There is no information linking these homologs to cell division.

### EXAMPLE 5

Characterization of the C. Elegans Gene K12D12.2

dsRNA was designed and used to specifically silence the expression of the C. elegans gene by RNAi, thereby testing its functional involvement in the first 2 rounds of embryonic cell division in this metazoan species. The dsRNA was synthesized in vitro from PCR-amplified wild type genomic DNA fragments of K12D12.2. For PCR, the following primer pair was used: “TAATACGACTCACTATAGGCGATAAGCACATAGCAGGCA” with “AATTAACCCTCACTAAAGGAAGGAGCTGTATCAAGCGGA” as forward and reverse primers, respectively. The dsRNA was purified, and injected into adult hermaphrodite worms. The phenotypic consequences of the RNAi treatment were documented 24 hours later in the F1 progeny of injected worms, using time-lapse differential interference contrast (DIC) microscopy. Embryo recordings started ˜20 minutes after ferilization, while the female pronucleus is completing its meiotic divisions, until the 4 cell stage, ˜30 minutes later.

In the F1 progeny of control worms that were either not injected, or injected with irrelevant dsRNA, the cellular events of the first two rounds of embryonic cell division were found to exhibit very limited variability, as observed by DIC microscopy and described in Example 3.

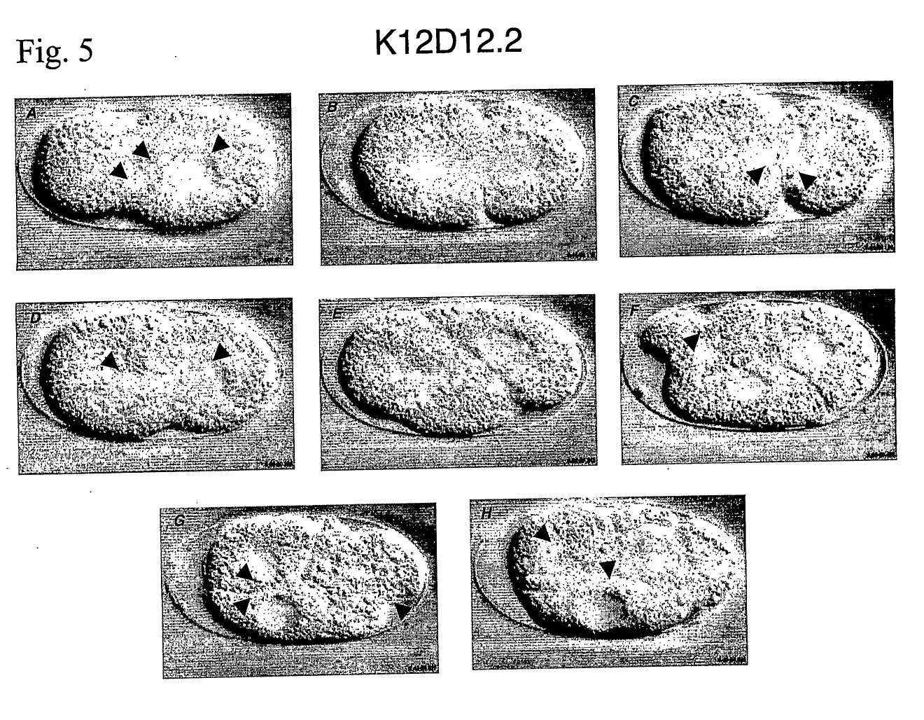

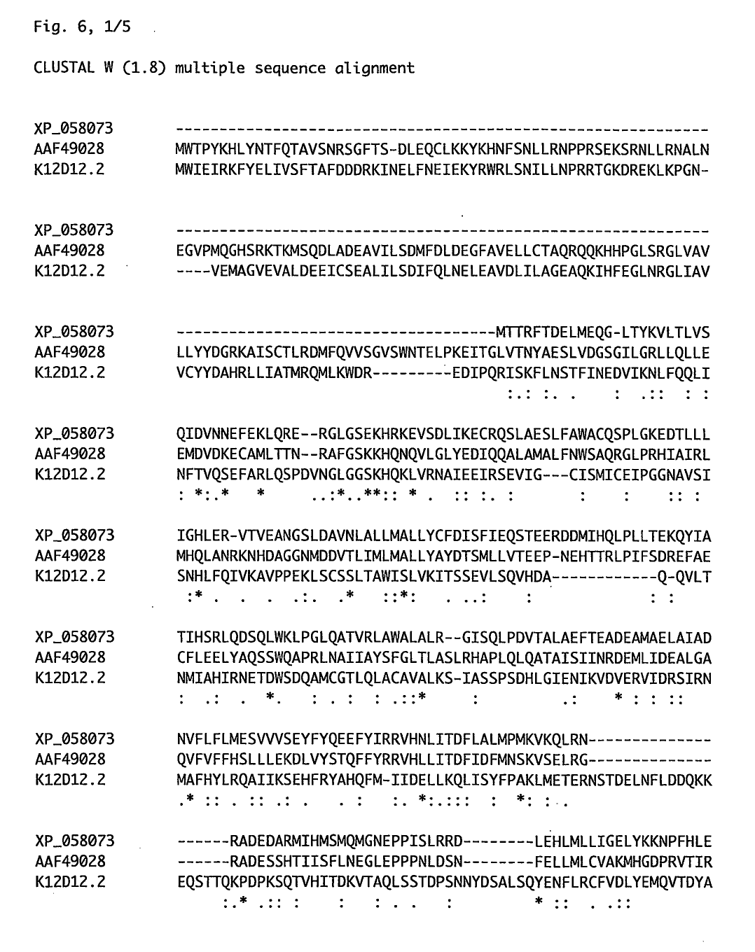

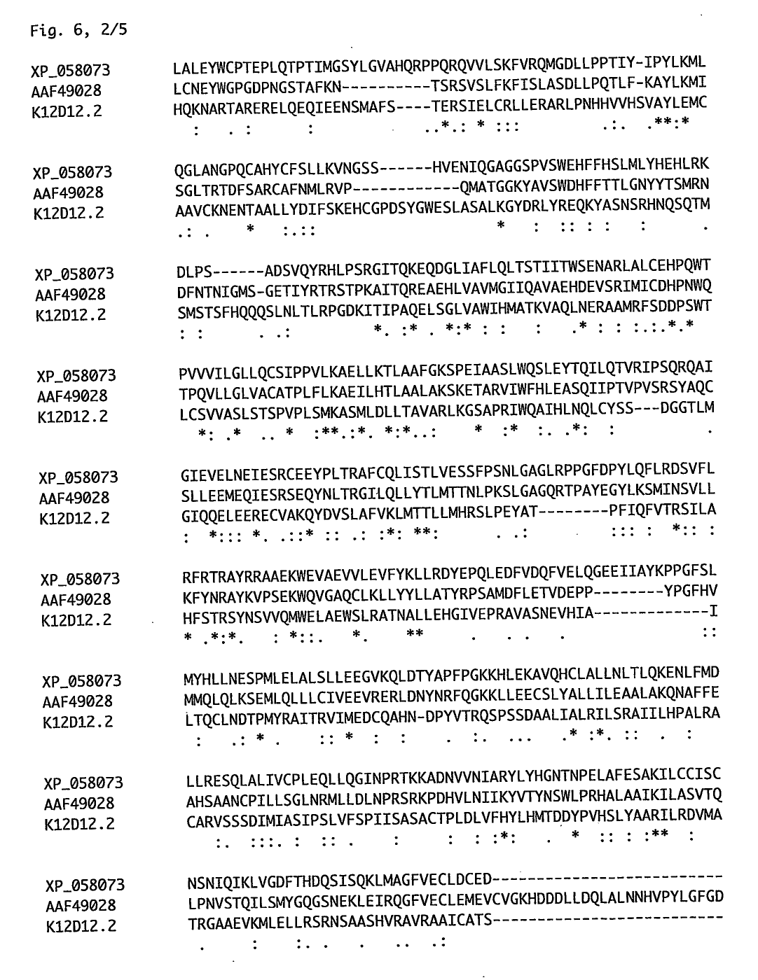

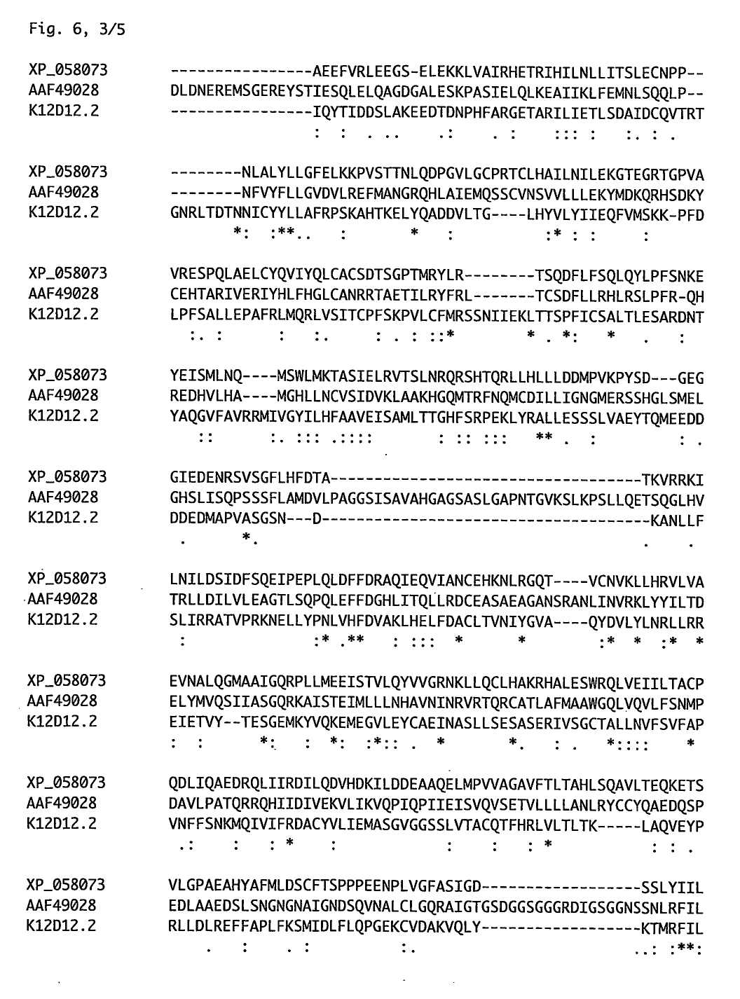

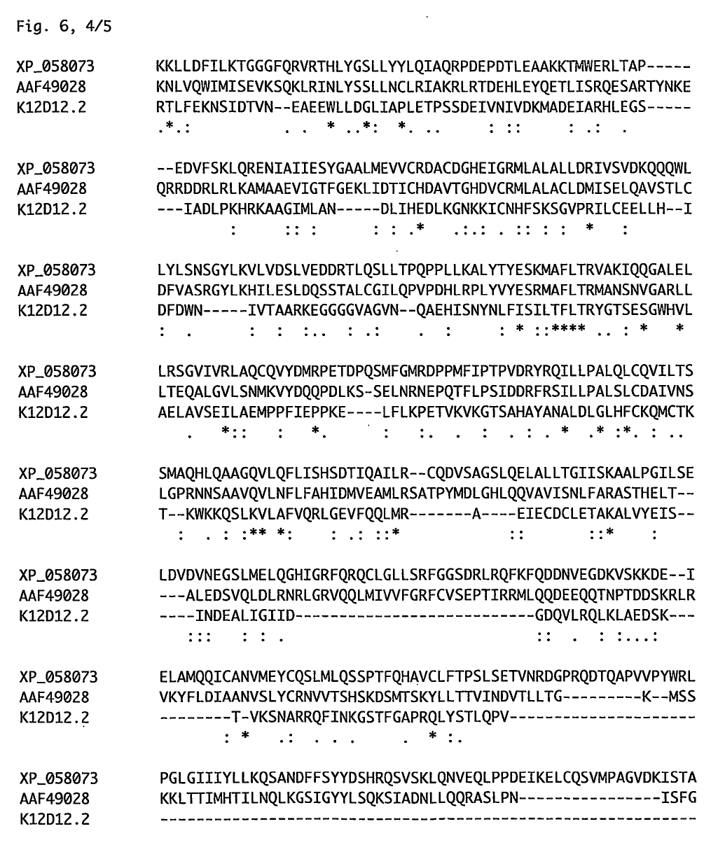

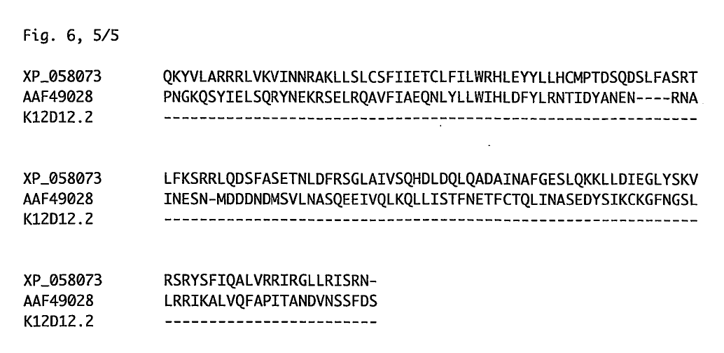

In the F1 embryos of worms injected with dsRNA, the following highly reproducible phenotypes are observed (FIG. 5). Three pronuclei are present (Fig. 5A, arrows), indicating a defect in meiosis. After cell division the nuclei stay attached to each other via a chromatin bridge, resulting in a tear-shaped appearance (FIG. 5C, D, arrows). Increased cortical activity in the AB cell (Fig. 5F, arrow). Division of the P1 cell is delayed (FIG. 5G, H).

All observed phenotypes indicate a requirement for K12D12.2 gene function in chromatin separation during meiosis and mitosis. Since this function is essential to cell division throughout metazoans, this gene and any homologs and derivatives thereof represent excellent tools for use in the development of a wide range of therapeutics including anti-proliferative agents. Analysis of the K12D12.2 gene sequence has revealed clear orthologs in human (GenBank Accession No. XP_{—}058073) and Drosophila (GenBank Accession No. AAF49028). There is no functional information available for these orthologs.

### EXAMPLE 6

Characterization of the C. Elegans Gene Y48E1B.12

dsRNA was designed and used to specifically silence the expression of the C. elegans gene by RNAi, thereby testing its functional involvement in the first 2 rounds of embryonic cell division in this metazoan species. The dsRNA was synthesized in vitro from PCR-amplified wild type genomic DNA fragments of Y48E1B.12. For the PCR, the following primer pair was. used: “TAATACGACTCACTATAGGCCACGCATTTTTCGATTTCT” with “AATTAACCCTCACTAAAGGAAAAATTAAAGCGGGGGAAA” as forward and reverse primers, respectively. The dsRNA was purified, and injected into adult hermaphrodite worms. The phenotypic consequences of the RNAi treatment were documented 24 hours later in the F1 progeny of injected worms, using time-lapse differential interference contrast (DIC) microscopy. Embryo recordings started ˜20 minutes after ferilization, while the female pronucleus is completing its meiotic divisions, until the 4 cell stage, ˜30 minutes later. In the F1 progeny of control worms that were either not injected, or injected with irrelevant dsRNA, the cellular events of the first two rounds of embryonic cell division were found to exhibit very limited variability, as observed by DIC microscopy and described in Example 3.

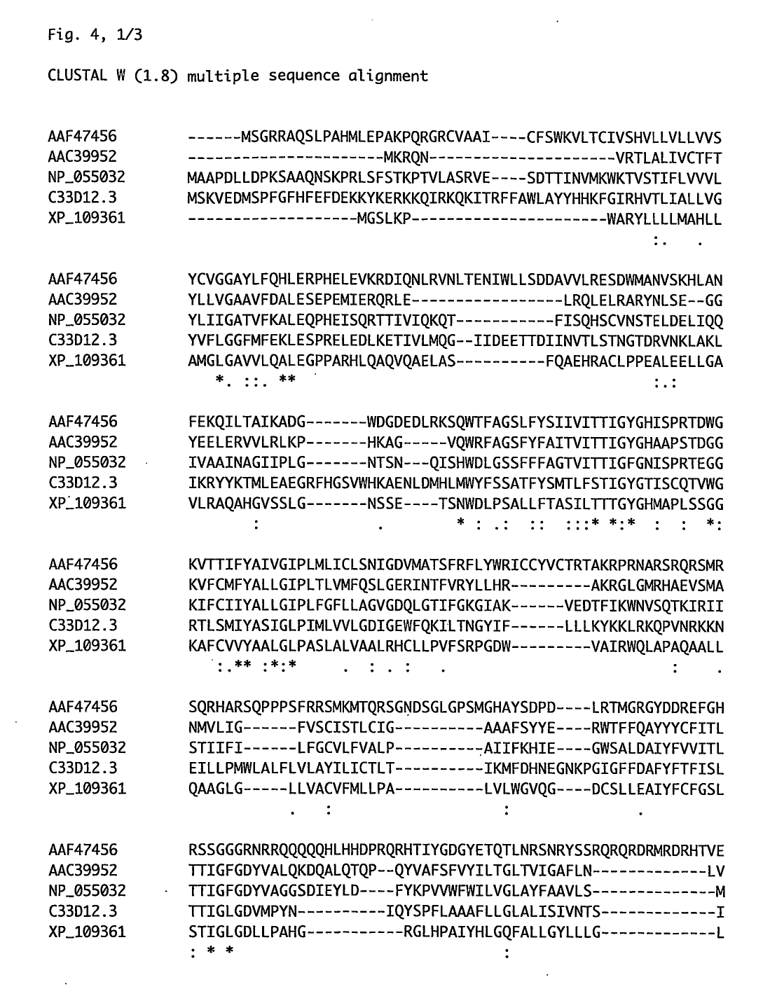

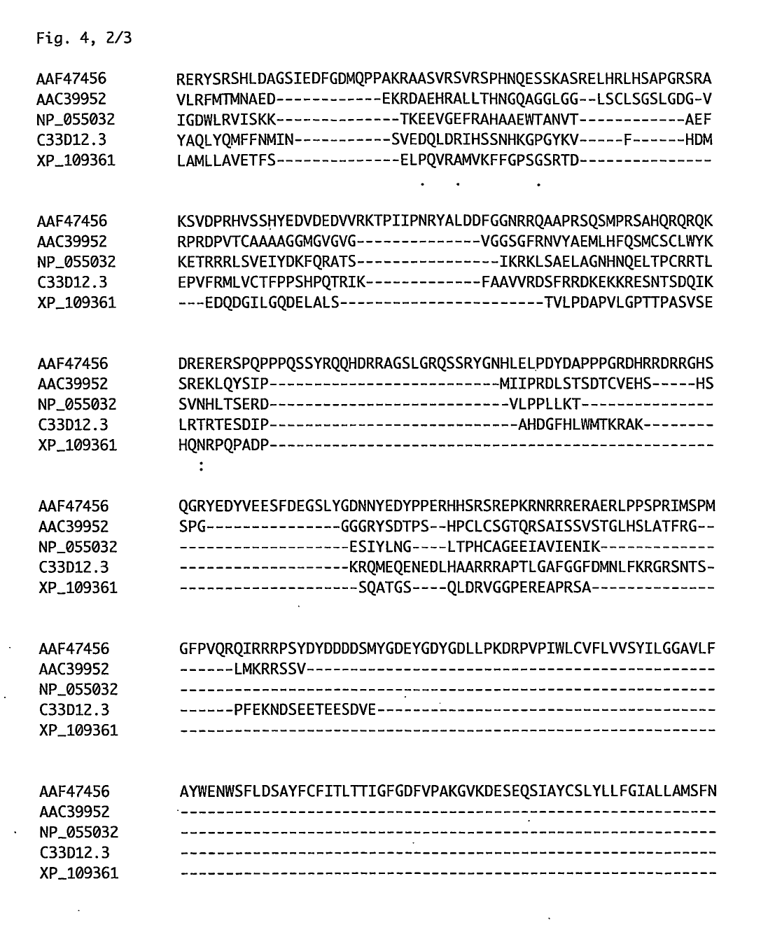

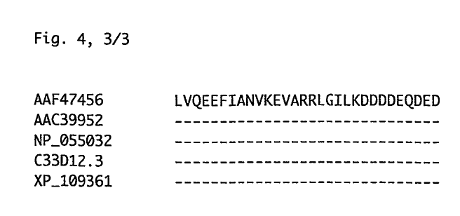

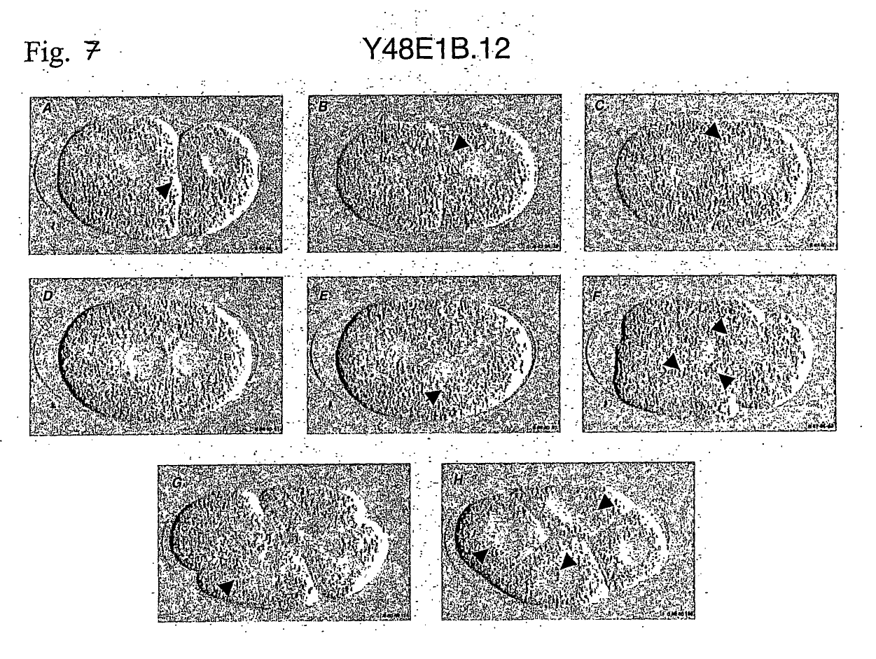

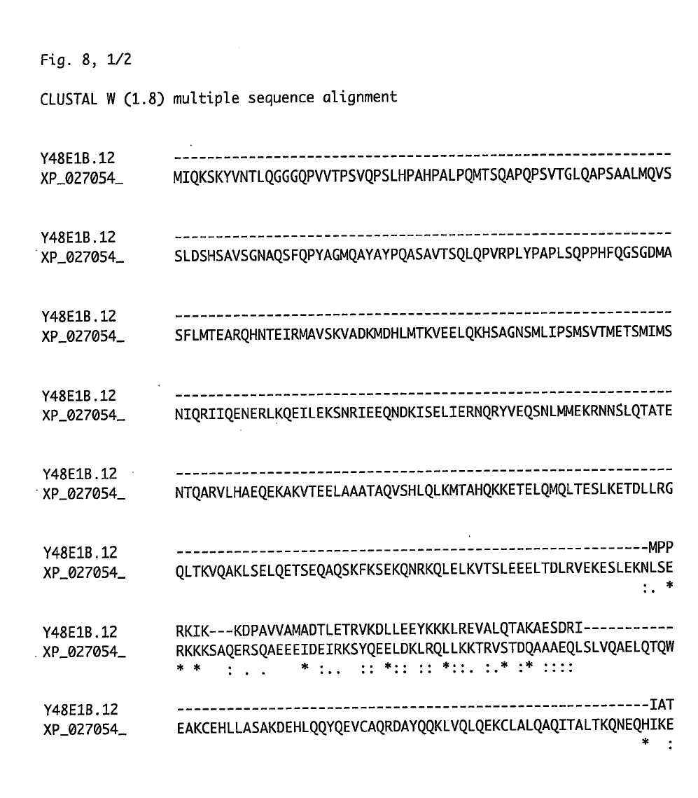

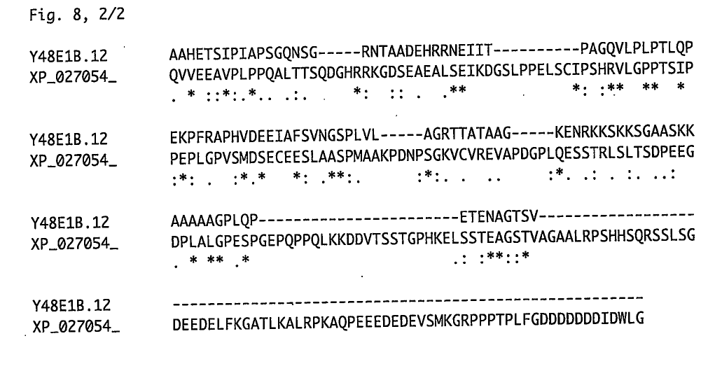

In the F1 embryos of worms injected with dsRNA, the following highly reproducible phenotypes are observed (FIG. 7). After cell division, the re-forming nuclei stay connected via a chromatin bridge, which results in failure of cytolinesis completion (FIG. 7A-D, arrow heads). The cleavage furrow regresses completely and a tetrapolar spindle is formed (FIG. 7E-F, arrow heads). Chromosome segregation is affected, mulitple karyomeres form in all blastomeres (FIG. 4G, H, arrow heads).

All observed phenotypes indicate a requirement for Y48E1B.12 gene function in chromatin separation during mitosis. Since this function is essential to cell division throughout metazoans, this gene and any hornologs and derivatives thereof represent excellent tools for use in the development of a wide range of therapeutics including anti-proliferative agents. Analysis of the Y48E1B.12 gene sequence has revealed a human gene showing significant homology (GenBank Accession No. XM_{—}027054). There is no functional information available for this human gene.

### EXAMPLE 7

Characterization of the C. Elegans Gene Y110A7A.1a

dsRNA was designed and used to specifically silence the expression of the C. elegans gene by RNAi, thereby testing its functional involvement in the first 2 rounds of embryonic cell division in this metazoan species. The dsRNA was synthesized in vitro from PCR-amplified wild type genomic DNA fragments of Y110A7A.1a. For the PCR, the following primer pair was used: “TAATACGACTCACTATAGGTGCAGTTCGTGAAAAACTCG” with “AATTAACCCTCACTAAAGGTTCAGATGCCACGTTGAGAG” as forward and reverse primers, respectively. The dsRNA was purified, and injected into adult hermaphrodite worms. The phenotypic consequences of the RNAi treatment were documented 24 hours later in the F1 progeny of injected worms, using time-lapse differential interference contrast (DIC) microscopy. Embryo recordings started ˜20 minutes after ferilization, while the female pronucleus is completing its meiotic: divisions, until the 4 cell stage, ˜30 minutes later.

In the F1 progeny of control worms that were either not injected, or injected with irrelevant dsRNA, the cellular events of the first two rounds of embryonic cell division were found to exhibit very limited variability, as observed by DIC microscopy and described in Example 3.

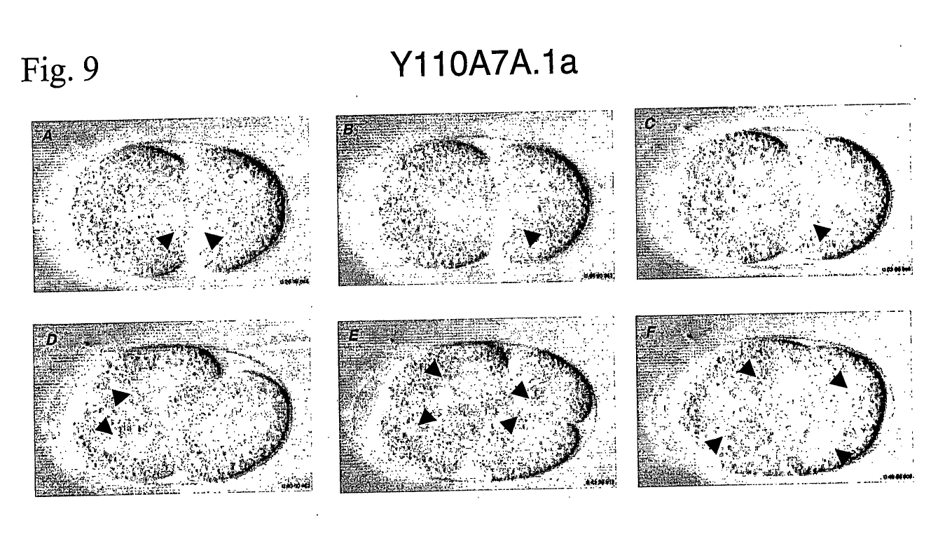

In the F1 embryos of worms injected with dsRNA, the following highly reproducible phenotypes are observed (FIG. 9). After cell division, the re-forming nuclei stay attached to the central cortex via a chromatin bridge. (FIG. 9A-C, arrow heads). Mulitiple small karyomeres are formed after the next round of mitosis (FIG. 9D-F, arrow heads).

All observed phenotypes indicate a requirement for Y110A7A.1a gene function in chromatin separation during meiosis and mitosis. Since this function is essential to cell division throughout metazoans, this gene and any homologs and derivatives thereof represent excellent tools for use in the development of a wide range of therapeutics including anti-proliferative agents. Analysis of the Y110A7A.1a gene sequence has revealed a clear ortholog in human (GenBanl Accession No. XM_{—}166201). There is no functional information available for these orthologs.

### EXAMPLE 8

Characterization of the C. Elegans Gene W02D9.2

dsRNA was designed and used to specifically silence the expression of the C. elegans gene by RNAi, thereby testing its functional involvement in the first 2 rounds of embryonic cell division in this metazoan species. The dsRNA was synthesized in vitro from PCR-amplified wild type genomic DNA fragments of W02D9.2. For PCR, the following primer pair was used: “TAATACGACTCACTATAGGTTTCAGCTGGAGCAGGAGAT” with “AATTAACCCTCACTAAAGGGAAGTGATGAGAAGCCGGAG” as forward and reverse primers, respectively. The dsRNA was purified, and injected into adult hermaphrodite worms. The phenotypic consequences of the RNAi treatment were documented 24 hours later in the F1 progeny of injected worms, using time-lapse differential interference contrast (DIC) microscopy. Embryo recordings started ˜20 minutes after ferilization, while the female pronucleus is completing its meiotic divisions, until the 4 cell stage, ˜30 minutes later.

In the F1 progeny of control worms that were either not injected, or injected with irrelevant dsRNA, the cellular events of the first two rounds of embryonic cell division were found to exhibit yery limited variability, as observed by DIC microscopy and described in Example 3.

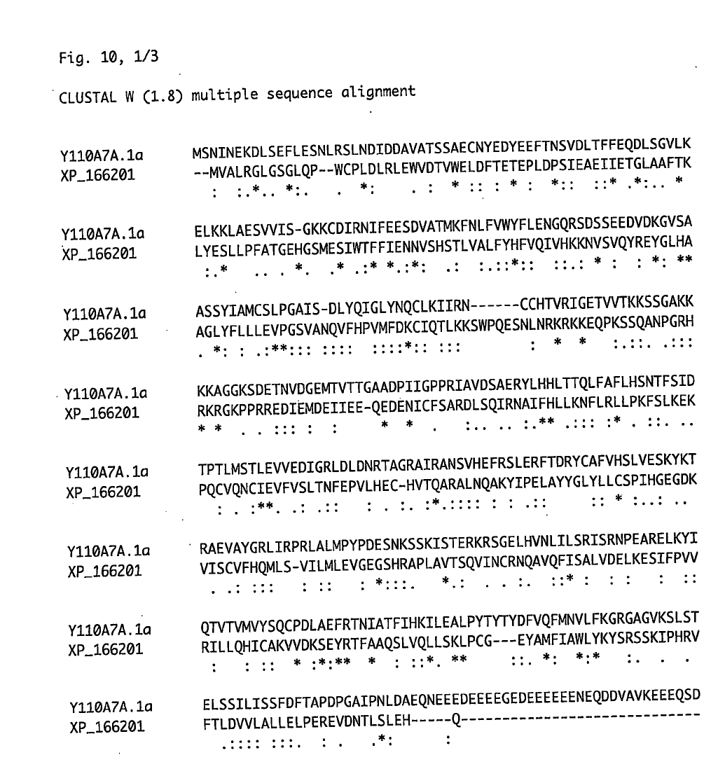

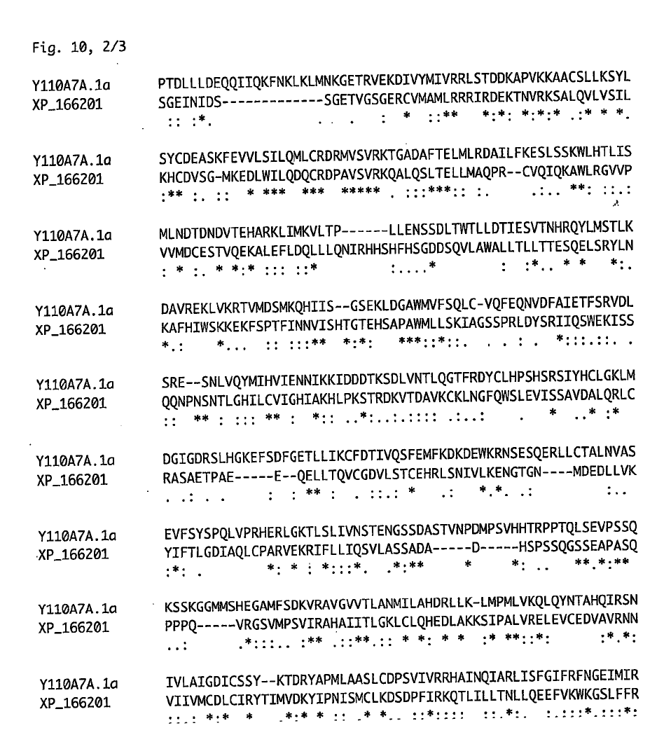

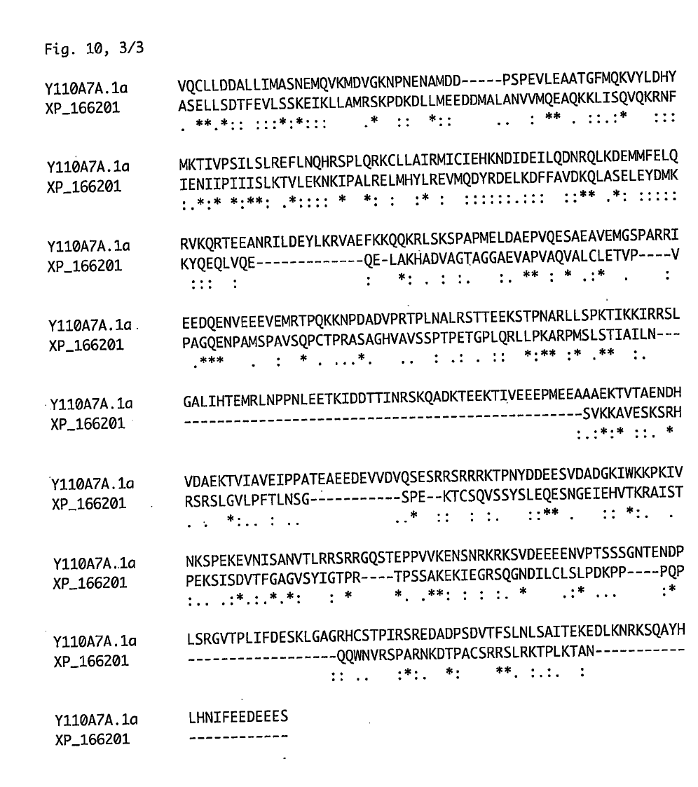

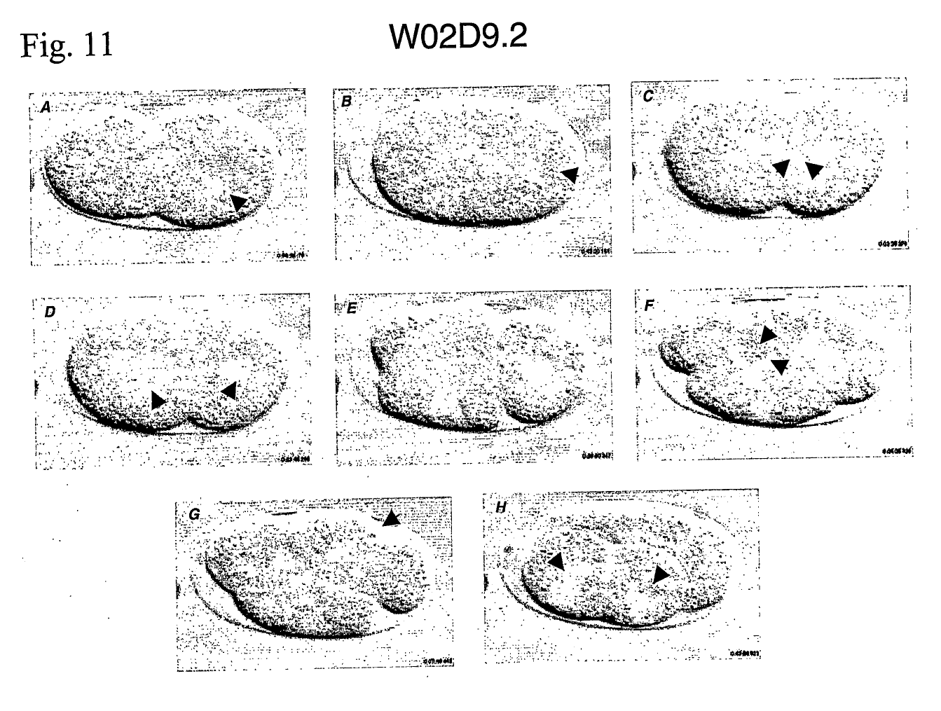

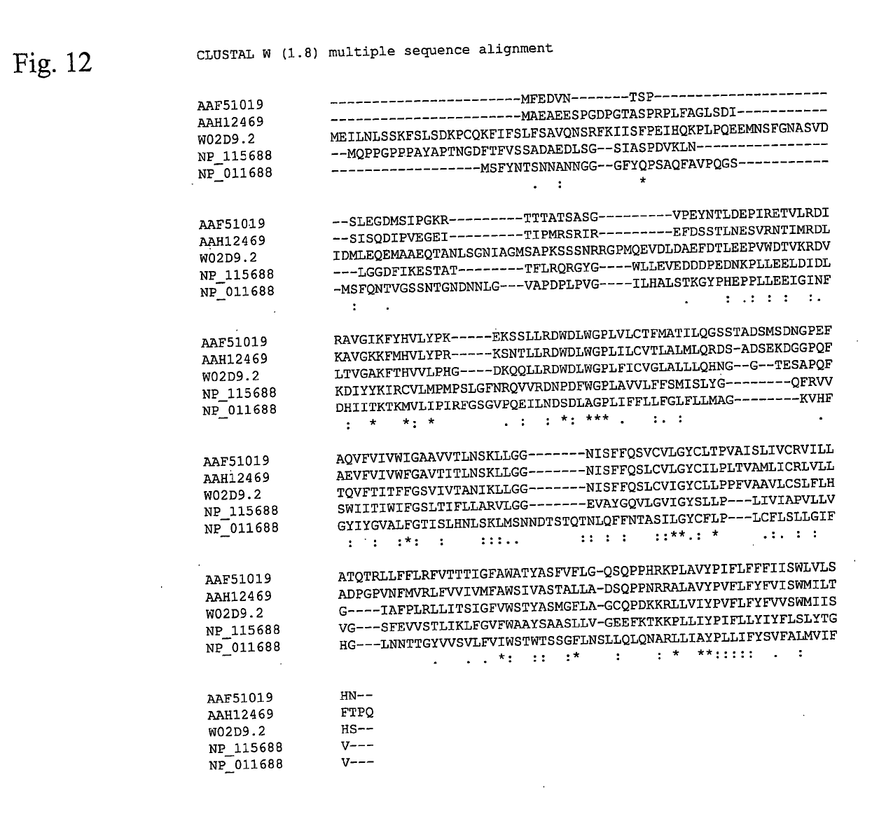

In the F1 embryos of worms injected with dsRNA, the following highly reproducible phenotypes are observed (FIG. 11). Jerky migration of the male pronucleus towards the female (FIG. 11A, B). After cell division, the re-forming nuclei stay connected via a chromatin bridge, resulting in a deformed appearance (FIG. 11 C-D, arrow heads). The same pattern emerges after the next division in the AB daughter cells (FIG. 11F, H, arrow heads).

All observed phenotypes indicate a requirement for W02D9.2 gene function in chromatin separation during meiosis and mitosis. Since this function is essential to cell division throughout metazoans, this gene and any homologs and derivatives thereof represent excellent tools for use in the development of a wide range of therapeutics including anti-proliferative agents. Analysis of the W02D9.2 gene sequence has revealed clear orthologs in Drosophila (GenBank Accession No. AAF51019) and human (GenBank Accession No. AAH12469), and homologs in human (GenBank Accession No. NP_{—}115688) and Saccharomyces cerevisiae (GenBanlc Accession No. NP_{—}011688). There is no information regarding these orthologs or homologs having a role in cell division.

### EXAMPLE 9

Protocol for Identifying Functional Orthologs in Other Species

To identify orthologous genes, the following procedure was used: The identified homologous amino acid sequences themselves were used for BLAST searches. If the original C. elegans protein was (re-)identified by a BLAST hit with an e-value of less than 10⁻⁵, the identified homolog was defined as an ortholog. The BLAST search was performed with the default parameters and the low complexity filter on. An alternative parameter for identification of homologous genes can be the percentage of sequence identity. Over 100 residues, a sequence identity of 30% defines a homologous gene. After the BLAST search is completed, multiple sequence alignment is performed using appropriate software (for example, CLUSTALW) and a neighbor joining phylogenetic tree is generated. Any person skilled in the art can identify the human ortholog from a phylogenetic tree. Essentially, the human sequence that is separated on the tree by a single speciation event or most closely related on the tree is likely to be an ortholog.

### EXAMPLE 10

Effects of RNAi Treatment in Human Cells

Design and Synthesis of siRNAs

For all experiments in human cells short double stranded interfering RNAs (siRNAs) of 21 bases in length, comprised of a 19 bp core of complementary sequence and 2 bases overhang at the 3′ end, were designed by Cenix and chemically synthesized by Ambion Inc., Austin, Tex., USA.

The following siRNA sequences were used:

| | |
|---|---|
| scrambled negative | 5-AGUACUGCUUACGAUACGGTT-3 |
| control | 3-TTUCAUGACGAAUGCUAUGCC-5 |
| positive control | 5-GGAGAAAGUUUCAGACUAUTT-3 |
| (PCNA, proliferating | 3-GTCCUCUUUCAAAGUCUGAUA-5 |
| cell nuclear antigen) | |
| AXH12469 | 5-GGCCUUUCAGAUAUAUCCATT-3 |
| | 3-GTCCGGAAAGUCUAUAUAGGU-5 |
| XP_166201.1 | 5-GGAGAGAAGAUAUUGAGAUTT-3 |
| | 3-GTCCUCUCUUCUAUAACUCUA-5 |
| NP_055032.1 | 5-GGCGGCAAAAUAUUCUGUATT-3 |
| | 3-TTCCGCCGUUUUAUAAGACAU-5 |
| NP_060230.2 | 5-GGUAGAAAAAACACAAAUGTT-3 |
| | 3-CTCCAUCUUUUUUGUGUUUAC-5 |
| XP_027054 | 5-GGCACCUUCUGCUGCCUUATT-3 |
| | 3-GTCCGUGGAAGACGACGGAAU-5 |
| XP_058073.3 | 5-GGUUUGGGCAGUGAAAAACTT-3 |
| | 3-CTCCAAACCCGUCACUUUUUG-5 |

Transfection

HeLa cells were treated with siRNAs at a final concentration of 100 nM using a lipofection based transfection protocol. 24 h before transfection, HeLa cells were seeded in 96well plates at a density of 6,000 cells/well.

On the day of transfection, the transfection mix was prepared as follows: 1 μl of a 10 μM stock of siRNA was diluted with 16 μl of Opti-MEM (Invitrogen Inc.), and 0.4 μl Oligofectamine transfection reagent (Invitrogen) were diluted with 2.4 μlof Opti-MEM. For complex formation, both solutions were gently mixed and incubated for 20 min at RT. Culture medium was removed from the cells and 80 μl of fresh medium (DMEM, Invitrogen) were added, followed by addition of 20 μl of transfection mix. Cells were incubated at 37° C. for 4 hours, 50 μl of fresh medium, supplemented with 30% fetal calf serum were added, followed by another incubation for 48-72 hours.

Determination of Silencing Level by Quantitative RT-PCR (qRT-PCR)

48 hours after transfection, total RNA was extracted from RNAi treated cells using Invisorb kits (Invitek GmbH, Berlin), and cDNA was produced with ABI TaqMan reverse transcription reagents (Applied Biosysteins, USA). In both cases the manufacturer's instructions were followed. Quantitative real-time PCR was performed using the following protocol: 5.5 μl of 2×SybrGreen PCR mix (Applied Biosystems) were mixed with 3 μl of sample cDNA and 2.5 μl of a 2 μM solution of gene specific PCR primers, followed by incubation in a ABI-7900-HT real-time PCR machine at 50° C. 2 min-95° C. 10 min-45 cycles (95° C. 15 sec-60° C. 1 min)-95° C. 15 sec-60° C. 15 sec-95°C. 15 sec. In addition to the gene specific reaction, a second, reference reaction was run for each cDNA sample, using primers for 18S rRNA. Amplification signals from different gene specific samples were normalized using the reference values on 18S rRNA for these respective samples, and compared to samples from control (scrambled siRNA from Ambion Inc.) treated cells.

Proliferation Assay

In order to quantify the number of living cells after RNAi treatment, ATP levels were measured 72h after transfection using the ATPlite assay (Perkin Elmer). Cells were extracted and treated according to the manufacturer's instructions. Luminescence read out was performed on a Victor 2 multi label reader (Perlcdn Elmer). For graphical presentation purposes the proliferation of untreated cells was set to 100.

Apoptosis Assay

The levels of programmed cell death in RNAi treated cells were determined 72 hours after transfection, using the Caspase 3/7 specific fluorometric assay ApoOne by Promega, following the manufacturer's instructions. Read out was performed on a Victor 2 multi label reader (Perkin Elmer). For graphical presentation purposes the apoptosis rate of untreated cells was set to 100.

Mitotic Index (MI)

Phosphorylation at serin 10 of histone H3 is considered a hallmark of mitosis, appearing in early prophase and disappearing during telophase. Using immunofluorescence microscopy, mitotic cells can be revealed by an increased binding of a phospho-histone H3 antibody, detected by a suitable fluorescence labelled secondary antibody.

RNAi treated cells in 96 well rnicroscopy plates were stained using the following protocol: Cells were washed with PBS and fixed with 4% para-formaldehyde for 30 min at RT, followed by three washes with PBS. Cells were then permeabilised and blocked in the presence of 0.1% Triton X-100 and 2% BSA for 30 min. The supernatant was removed and anti Phospho Histone H3 (mouse monoclonal antibody clone 6G3, Cell Signalling Technologies) was added at a dilution of 1:750 for 2 hours at RT, followed by three washes with PBS. For detection of Phosph Histone H3 labelled nuclei, goat anti mouse antibody (1:500), coupled to Alexa Fluor 568 (Molecular Probes) was added in a solution supplemented with 0.5 μg/ml Dapi (4′,6-diamidino-2-phenylindole, dihydrochloride), FlubroPure™ grade, Molecular Probes) for detection of all nuclei. After incubation for 2 hours at RT, cells were washed four times and images were taken using an automated microscopy system (Discovery-1, Universal Imaging Inc.), acquiring a minimum of 6 images/well. Metamorph-HCS image processing software was used to determine the numbers of mitotic and overall nuclei. The Mitotic Index resembles the fraction of mitotic over all nuclei in a given cell population. For graphical presentation purposes the MI of untreated cells was set to 100.

Effects of RNAi Treatment

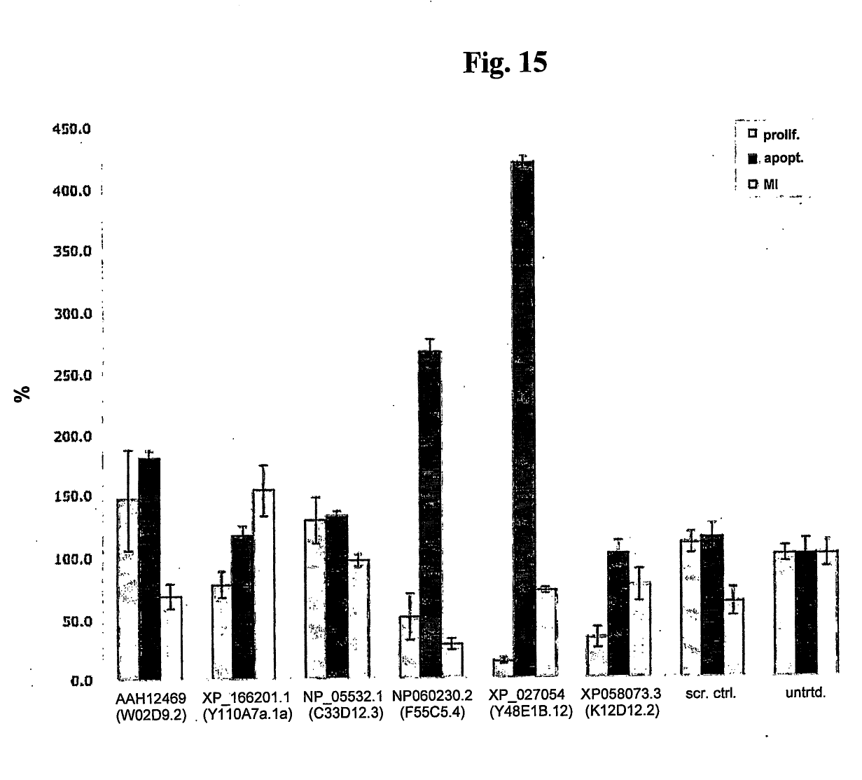

As shown in FIG. 15, RNAi treatment of HeLa cells using an siRNA directed against AAH12469, the human ortholog of C. elegans gene W02D9.2, results in a significant increase in the rate of apoptosis compared to control treated cells. RNAi treatment of HeLa cells using an siRNA directed against XP_{—}166201.1, the human ortholog of C. elegans gene Y110A7A.1a, results in a 25% reduction of cell proliferation and a 2fold increase in the Mitotic Index compared to control treated cells. RNAi treatment of HeLa cells using an siRNA directed against NP_{—}055032.1, the human ortholog of C. elegans gene C33D12.3, results in a subtle increase in the rate of apoptosis and an increase of the Mitotic Index compared to control treated cells. RNAi treatment of HeLa cells using an siRNA directed against NP_{—}060230.2, the human ortholog of C. elegans gene F55C5.4, results in a 50% reduction of cell proliferation, a 2.5fold increase in the rate of apoptosis and a 60% decrease in the Mitotic Index compared to control treated cells. RNAi treatment of HeLa cells using an siRNA directed against XP_{—}027054, the human ortholog of C. elegans gene Y48E1B.12, results in a 80% reduction of cell proliferation and a 4fold increase in the rate of apoptosis compared to control treated cells. RNAi treatment of HeLa cells using an siRNA directed against XP_{—}058073.3, the Human ortholog of C. elegans gene K12D12.2, results in a 70% reduction of cell proliferation compared to control treated cells.

1-25. (canceled)

26. A method for the inhibition of chromatin separation, comprising administering to a patient in need thereof an effective amount of a molecule selected from the group consisting of:
(1) an isolated nucleic acid molecule comprising a nucleic acid molecule with a sequence selected from the group of sequences consisting of:
(a) the nucleic acid sequences presented in SEQ ID NOs. 21, 23, 1, 3, 5, 7, 9, 11, 13, 15, 17, 19, 25, 27, 29, 31, 33, 35, 37;
(b) nucleic acid sequences encoding polypeptides that exhibit a sequence identity with the protein encoded by a nucleic acid according to (1)(a) of at least 25% over 100 residues and/or which are detectable in a computer aided search using the BLAST sequence analysis programs with an e-value of at most 10⁻⁵,
(c) sequences of nucleic acid molecules which are capable of hybridizing with the nucleic acid molecules with sequences corresponding to (1)(a) or (b) under conditions of medium or high stringency,
(d) the antisense-sequence of any of the sequences as defined in (1)(a), (b) or (c),
(e) fragments of (1)(a), (b), (c) or (d), and
(f) double-stranded RNA or single-stranded RNA in the antisense or sense direction corresponding to any of the sequences as defined in (1)(a), (b), (c), (d), or (e),
(2) an isolated peptide or polypeptide comprising a peptide or polypeptide with a sequence selected from the group consisting of:
(a) a sequence as disclosed in SEQ ID NOs. 22, 24, 2, 4, 6, 8, 10, 12, 14, 16, 18, 20, 26, 28, 30, 32, 34, 36, 38;
(b) a sequence that exhibits a sequence identity with any of the sequences according to (2)(a) of at least 25% over 100 residues, and
(c) fragments of the sequences defined in (2)(a) or (b),
and
(3) an antibody which is directed against at least one peptide or polypeptide with a sequence as defined in (2)(a) to (c) above.

27. The method according to claim 26, wherein the isolated nucleic acid molecule comprises small interfering RNA with a sequence corresponding to any of the sequences according to claim 26, or wherein the nucleic acid molecule is contained in at least one nucleic acid expression vector which is capable of producing a double-stranded RNA-molecule comprising a sense-RNA-strand and an antisense-RNA-strand under suitable conditions, wherein each RNA-strand, independently from the other, has a length of 19 to 31 nucleotides or wherein the nucleic acid molecule is contained in at least one nucleic acid expression vector comprising a first expression cassette containing the nucleic acid corresponding to the sense-RNA-strand under the control of a first promoter and a second expression cassette containing the nucleic acid corresponding to the antisense-RNA-strand under the control of a second promoter or wherein the nucleic acid molecule is contained in at least one nucleic acid expression vector comprising an expression cassette containing the nucleic acid corresponding to the sense-RNA-strand and the antisense-RNA-strand under the control of a promoter leading to a single-stranded RNA-molecule and wherein the single-stranded RNA-molecule is capable of forming a back-folded stem-loop-structure.

28. The method according to claim 27, wherein each RNA-strand, independently from the other, has a length of 20 to 25 nucleotides.

29. The method according to claim 27, wherein each RNA-strand, independently from the other, has a length of 26 to 28 nucleotides.

30. The method according to claim 26, wherein the method is for the treatment of a proliferative disease.

31. The method according to claim 30, wherein the disease is coronary restenosis or a neoplastic disease.

32. The method according to claim 31, wherein the neoplastic disease is selected from the group consisting of lymphoma, lung cancer, colon cancer, ovarian cancer and breast cancer.

33. A method for the activation of chromatin separation, comprising administering to a patient in need thereof an effective amount of a molecule selected from the group consisting of:
(1) an isolated nucleic acid molecule comprising a nucleic acid with a sequence selected from the group of sequences consisting of:
(a) the nucleic acid sequences presented in SEQ ID NOs. 21, 23, 1, 3, 5, 7, 9, 11, 13, 15, 17, 19, 25, 27, 29, 31, 33, 35, 37;
(b) nucleic acid sequences encoding polypeptides that exhibit a sequence identity with the protein encoded by a nucleic acid according to (1)(a) of at least 25% over 100 residues and/or which are detectable in a computer aided search using the BLAST sequence analysis programs with an e-value of at most 10⁻⁵,
(c) sequences of nucleic acid molecules which are capable of hybridizing with the nucleic acid molecules with sequences corresponding to (1)(a) or (b) under conditions of medium or high stringency,
(d) the antisense-sequence of any of the sequences as defined in (1 )(a), (b) or (c),
(e) fragments of (1l)(a), (b), (c) or (d), and
(f) RNA sequences corresponding to any of the sequences as defined in (1)(a), (b), (c), (d), or (e),
(2) an isolated peptide or polypeptide comprising a peptide or polypeptide with a sequence selected from the group consisting of:
(a) a sequence as disclosed in SEQ ID NOs. 22, 24, 2, 4, 6, 8, 10, 12, 14, 16, 18, 20, 26, 28, 30, 32, 34, 36, 38;
(b) a sequence that exhibits a sequence identity with any of the sequences according to (2)(a) of at least 25% over 100 residues, and
(c) fragments of the sequences defined in (2)(a) or (b),
and
(3) an antibody which is directed against at least one peptide or polypeptide with a sequence as defined in (2)(a) to (b) above.

34. The method according to claim 33, wherein the method is for the treatment of a disease characterized by increased apoptosis, growth retardation, or slowed wound healing.

35. A method for the in vitro diagnosis of a proliferative disease or a disease associated with abnormal chromatin separation, wherein the method comprises obtaining a sample tissue or cells thereof, and determining the amount of the nucleic acid molecule or of the protein or peptide as defined in claim 26 in the sample tissue or cells.

36. The method according to claim 35, wherein the disease is coronary restenosis or a neoplastic disease.

37. The method according to claim 36, wherein the neoplastic disease is selected from the group consisting of lymphoma, lung cancer, colon cancer, ovarian cancer and breast cancer.

38. A method for the identification and characterization of drugs that inhibit or activates chromatin separation, wherein the method comprises using the molecule as defined in claim 26 in a screening assay for the identification of said drug, and performing a functional analysis for the chromatin separation.
